(19) United States
(12) Patent Application Publication
(10) Pub. No.: US 2026/0183569 A1
(43) Pub. Date: Jul. 2, 2026

(54) ADJUSTABLE RADIOTHERAPY COLLIMATOR DEVICES AND SYSTEMS

(71) Applicant: BOARD OF REGENTS, THE UNIVERSITY OF TEXAS SYSTEM, Austin, TX (US)

(72) Inventors: Neil KIRBY, San Antonio, TX (US); Holly M. PASCHAL, Austin, TX (US)

(21) Appl. No.: 18/863,850

(22) PCT Filed: May 9, 2023
(86) PCT No.: PCT/US2023/021476

§ 371 Date: Nov. 7, 2024

Related U.S. Application Data
(60) Provisional application No. 63/339,752, filed on May 9, 2022.

(51) Int. Cl. A61N 5/10; G21K 1/04

(52) U.S. Cl. CPC A61N 5/1045; G21K 1/046

(57) ABSTRACT

Described herein are systems and devices for radiotherapy collimation. An example device includes a plurality of elongate members arranged in parallel to one another; and at least one fastener at least partially surrounding the plurality of elongate members, the at least one fastener being configured to apply compression to the plurality of elongate members, where: the plurality of elongate members are individually slidable in an uncompressed state, and the plurality of elongate members are individually fixed in a compressed state.

### BACKGROUND

Cancer tissues are often surrounded by healthy organs. The goal of radiation therapy is to target cancerous cells while minimizing the dose to non-cancerous tissue. This is accomplished by shaping radiation beams to target only cancerous tissues. In radiation beam shaping, radiation is collimated as it exits a particle accelerator. Radiation that is pointed at the tumor is transmitted and that which is pointed at normal tissue is blocked. One of the early methods for doing this is with Cerrobend, a castable metal alloy which can be shaped for each individual patient, is toxic and requires each collimator to be formed individually.

Another beam shaping technique is known as a multi-leaf collimator (MLC). Instead of creating custom collimation for each patient (like the Cerrobend collimators), a system of motors is used to move thick collimating leaves to shape the radiation. MLC shapes can be digitally designed and then be ready for delivery at the treatment machine. Additionally, a combination of MLC fields can be used to create more complex radiation patterns, which can spare normal tissue while treating a tumor. More specifically, these techniques are referred to as intensity-modulated radiation therapy (IMRT) and volumetric modulated arc therapy (VMAT).

Photons and electrons are the commonly available types of particles for delivering radiotherapy. Electrons have a limited range in tissue and can be used to treat targets at shallow tissue depths and also enables them to minimize dose delivered beyond the target. One of the other major differences between these types of radiation is how they scatter. Photons will effectively travel in a straight line through air, whereas electrons are easily scattered in air. This can impact collimation of these particles. For photons, a beam can be collimated far away from a patient and that shape will hold while traveling to the patient. In contrast, electron scattering will quickly smear out a radiation pattern as it travels through air. This can affect how quickly radiation dose falls off outside of a target. Although MLCs are the standard for photon therapy, Cerrobend collimation is still the standard for electrons.

Additionally, radiation can scatter or “smear” as it passes through air. As a result, when a collimator is placed further from a patient (i.e. the treatment surface), the amount of scattering or smearing can increase. Therefore, what is required are systems and methods directed to these and other considerations.

### SUMMARY

Systems, devices, and methods for electron collimation are described herein.

An example device for radiotherapy collimation is described herein. The device can include a plurality of elongate members arranged in parallel to one another; and at least one fastener at least partially surrounding the plurality of elongate members. The at least one fastener is configured to apply compression to the plurality of elongate members, where: the plurality of elongate members are individually slidable in an uncompressed state, and the plurality of elongate members are individually fixed in a compressed state.

In some implementations, the radiotherapy collimation device is flexible and configured to contort into a collimator shape in the uncompressed state. Alternatively or additionally, the radiotherapy collimation device is inflexible and fixed in the collimator shape in the compressed state.

In some implementations, the collimator shape is a two-dimensional shape. Alternatively or additionally, the collimator shape is a three-dimensional shape. Optionally, the three-dimensional shape is a helix.

In some implementations, the at least one fastener is a plurality of fasteners, each of the plurality of fasteners at least partially surrounding the plurality of elongate members. Optionally, in some implementations, the at least one fastener is a clamp. Optionally, the clamp is adjustable to change a circumference of the clamp.

Alternatively or additionally, the at least one fastener is a vacuum-tight-sleeve. Optionally, the vacuum-tight-sleeve is a flexible tubing, and where each of the plurality of elongate members is arranged at least partially inside the flexible tubing. Optionally, the flexible tubing includes an elastic material. In some implementations, the flexible tubing is configured to apply pressure to the plurality of elongate members in response to a change in pressure within the flexible tubing.

In some implementations, each of the plurality of elongate members is a metal cable.

In some implementations, the radiotherapy collimation device includes a locking mechanism configured to attach to an end of the plurality of elongate members. Optionally, in some implementations, the radiotherapy collimation device has a first end and a second end, each of the first and second ends defined by respective ends of the plurality of elongate members. In these implementations, the radiation collimation device further includes a locking mechanism configured to attach the first end to the second end of the radiotherapy collimation device.

An example system for performing radiotherapy is described herein. The system can include: a radiation source configured to emit a beam of electron radiation along a beam axis toward a subject; a radiotherapy collimation device as described herein, where the radiotherapy collimation device is configured for forming the beam into a desired beam shape in proximity to the subject.

In some implementations, the plurality of elongate members define a longitudinal axis, and the longitudinal axis is perpendicular to the beam axis.

In some implementations, the radiotherapy collimation device is attached in a fixed position relative to the radiation source. Optionally, the radiotherapy collimation device is arranged in a fixed position relative to the subject.

An example multi-leaf collimation device is described herein. The device can include a leaf guide defining an aperture plane, a first axis, and a second axis, where the first axis is perpendicular to the second axis, and where the second axis is perpendicular to the aperture plane; a plurality of leaves arranged within the leaf guide; and one or more leaf drivers operably connected to the plurality of leaves. The one or more leaf drivers are configured to move the plurality of leaves to form a radiation-beam aperture in the aperture plane, and at least one leaf is moveable along the second axis to extend beyond the aperture plane in a direction toward a subject.

In some implementations, the at least one leaf includes a first section, a second section, and a curved section, where the first and second sections are separated by the curved section. Optionally, the curved section defines about a 90 degree angle between the first and second sections.

In some implementations, the one or more leaf drivers are configured to move the at least one leaf along the first and second axes.

In some implementations, the at least one leaf is made of a biocompatible material. Optionally, the at least one leaf includes plastic. Alternatively or additionally, the at least one leaf includes steel, aluminum, or brass. Alternatively or additionally, the at least one leaf is made of a material that minimizes x-ray production when absorbing electrons.

In some implementations, the one or more leaf drivers are configured to move the plurality of leaves independently from each other.

In some implementations, the leaf guide includes a first leaf guide comprising a first plurality of leaves and a second leaf guide comprising a second plurality of leaves, where the one or more leaf drivers are configured to move the first and second plurality of leaves to form the radiation-beam aperture in the aperture plane.

An example radiotherapy system is described herein. The radiotherapy system can include a multi-leaf collimation device as described herein; and a radiation source configured to emit a beam of electron radiation along the second axis toward a subject, where the multi-leaf collimation device is configured for forming the beam into a desired beam shape in proximity to the subject.

An example radiotherapy collimation device is described herein. The radiotherapy collimation device can include plurality of elongate members arranged in parallel to one another and forming a plurality of loops; and at least one fastener configured to apply compression to the plurality of elongate members, where: the plurality of elongate members are individually slidable in an uncompressed state, and the plurality of elongate members are individually fixed in a compressed state.

In some implementations, the at least one fastener includes a hub configured to pass through at least one of the plurality of loops and a cap configured to clamp the hub to at least one of the plurality of loops.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates a system for performing therapy using electron radiation, according to implementations of the present disclosure.

FIGS. 2A-2C illustrate perspective views of a flexible collimator, according to an implementation of the present disclosure. FIG. 2A illustrates a first perspective view of a flexible collimator, and FIG. 2B illustrates a second perspective view of the flexible collimator shown in FIG. 2A. FIG. 2C illustrates the flexible collimator of FIGS. 2A-2B contorted into a curved shape.

FIGS. 3A-3C illustrate a flexible collimator used to treat a tumor, according to an implementation of the present disclosure. In FIGS. 3A-3C, the flexible collimator is a coil that forms a helix. FIG. 3A illustrates a top view of the tumor through an aperture of the flexible collimator. FIG. 3B illustrates a cutaway side view of the flexible collimator illustrated in FIG. 3A. FIG. 3C illustrates a perspective view of the flexible collimator shown in FIGS. 3A and 3B.

FIGS. 4A-4C illustrate a multi-leaf collimator (MLC). FIG. 4A illustrates a block diagram of an MLC system. FIG. 4B illustrates a multi-leaf collimator that does not conform to the patient surface. FIG. 4C illustrates the movement direction of the leaves in the multi-leaf collimator of FIG. 4B. FIG. 4D illustrates a multi-leaf collimator that conforms to the patient surface, according to an implementation of the present disclosure.

FIGS. 5A-5B illustrate an experiment to test an example implementation of the present disclosure. FIG. 5A illustrates the experimental setup. FIG. 5B illustrates a test result from the experiment illustrated in FIG. 5A.

FIG. 6 illustrates experimental results of a comparison of the measured 6 MeV radiation from film representing the dose falloff on skin surface from a conventional collimator compared to that from an example implementation of the present disclosure.

FIG. 7 illustrates a table of experimental results from an example implementation of the present disclosure including penumbra measurements at the surface and at depth for 6 and 15 MeV where the tumor depth was measured as 12 and 27 mm for 6 and 15 MeV, respectively.

FIG. 8 illustrates an example implementation of the present disclosure including a flexible collimator using a vacuum sleeve.

FIGS. 9A-9C illustrate images of an example implementation of an electron MLC. FIG. 9A illustrates a roller bearing “skate” system for MLC motion; FIG. 9B illustrates four MLCs in a bank that can be moved normal relative to a patient surface. FIG. 9C illustrates an example implementation of an electron MLC including 32 MLCs.

FIG. 10 illustrates a dose profile at the depth of a tumor for a 6 MeV electron beam collimated with the example implementation of the present disclosure illustrated in FIG. 9C.

FIGS. 11A-11B illustrate experimental results of a comparison of 6 MeV isodose distributions. Percentages are relative to the maximum dose. FIG. 11A illustrates the isodose distribution for Cerrobend, and FIG. 11B illustrates the isodose distribution of an example implementation of the present disclosure.

FIGS. 12A-12B illustrate experimental results of a comparison of 15 MeV isodose distributions. Percentages are relative to the maximum dose. FIG. 12A illustrates the isodose distribution for Cerrobend, and FIG. 12B illustrates the isodose distribution of an example implementation of the present disclosure.

FIG. 13 illustrates experimental results of a comparison of penumbra size (defined as the distance between 20% and 80% of central axis dose) and dose transmission 3 cm outside the field edge for the surface conforming electron MLC (SCEM) and Cerrobend cutout for a 10×10 cm field at dₘₐₓ for different energies.

FIG. 14 illustrates an example cable collimator with looped ends, according to an implementation of the present disclosure.

FIG. 15 illustrates a leaf of a multi leaf collimator, according to an implementation of the present disclosure.

FIG. 16A illustrates a top view of a number of leaves of an MLC arranged into banks, according to an implementation of the present disclosure.

FIG. 16B illustrates a perspective view of a number of leaves of an MLC arranged into banks, according to an implementation of the present disclosure.

FIG. 17 illustrates percent depth dose (PDD) curves for the SCEM (dashed) and Cerrobend cutouts (solid) where the y-axis is the central-axis percent depth dose relative to the maximum and the x-axis is the central axis depth in millimeters, according to an example implementation of the present disclosure.

FIG. 18 illustrates a table of surface dose (% relative to dmax) and the bremsstrahlung tail (i.e. the central axis dose relative to dmax 1 cm beyond the practical range) for the Cerrobend cutout at 100 cm SSD and the SCEM according to an example implementation of the present disclosure.

FIG. 19 illustrates acomparison of the isodose lines for the cerrobend cutout at 100 cm SSD, cerrobend cutout at 110 cm SSD, and the SCEM for the 3×9 cm field size for each energy according to an example implementation of the present disclosure where the isodose lines shown from lowest to highest are 10%, 30%, 50%, 80%, amd 90%.

FIG. 20 illustrates a comparison of the isodose lines for the cerrobend cutout at 100 cm SSD, cerrobend cutout at 110 cm SSD, and the SCEM for the 5×9 cm field size for each energy according to an example implementation of the present disclosure where the isodose lines shown from lowest to highest are 10%, 30%, 50%, 80%, and 90%.

FIG. 21 illustrates a comparison of the isodose lines for the cerrobend cutout at 100 cm SSD, cerrobend cutout at 110 cm SSD, and the SCEM for the 10×9 cm field size for each energy according to an example implementation of the present disclosure where the isodose lines shown from lowest to highest are 10%, 30%, 50%, 80%, amd 90%.

FIG. 22 illustrates a comparison of penumbra size (defined as the distance between 20% and 80% of central axis dose) at dmax for each field size and energy according to an example implementation of the present disclosure.

FIG. 23 ilustrates a omparison of dose transmission 3 cm outside the field edge for the SCEM and Cerrobend cutout (100 cm SSD and 110 cm SSD) at dmax for all field sizes and energies according to an example implementation of the present disclosure.

FIG. 24 illustrates Output factors for each energy for the SCEM (dashed) and Cerrobend cutouts (solid), normalized to the 10×9 cm field size according to an example implementation of the present disclosure.

FIG. 25 illustrates MiniPhantom output factors for each energy for the SCEM (dashed) and Cerrobend cutouts (solid), normalized to the 10×9 cm field size according to an example implementation of the present disclosure.

FIG. 26 illustrates a table of ratios of raw charge readings (SCEM to Cerrobend cutout) for the 10×9 cm field for all energies.

FIG. 27 illustrates cross-plane profiles for the SCEM for the 10×9 cm field size at 1 mm depth (near-surface) and dmax for each energy

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the reference list. For example, Ref. [1] refers to the 1^{st} reference in the list. All references cited and discussed in this specification are incorporated herein by reference in their entireties and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

While the present disclosure references “tumors” or “cancer” as the target for treatment, it should be understood that implementations of the present disclosure can be used to collimate radiation for any radiotherapy treatment, e.g. the removal of other types of lesions.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

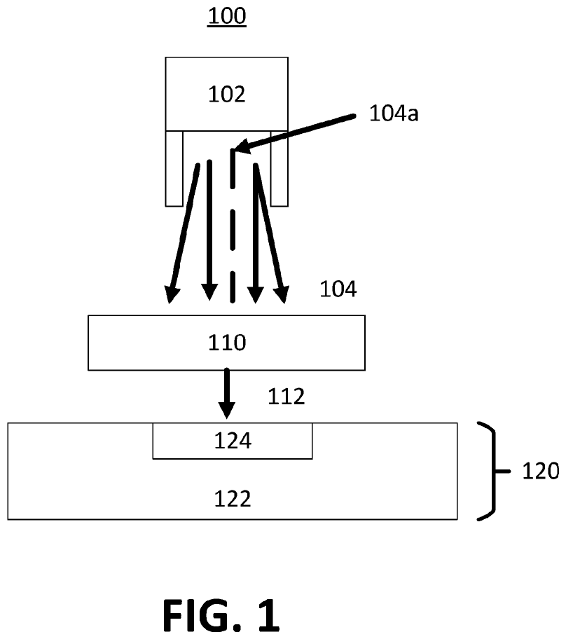

Implementations of the present disclosure are directed to systems and devices that can be used to collimate radiation beams used in radiotherapy. In the examples described herein, the collimation devices are used to collimate electron beams, which are susceptible to scattering or smearing. It should be understood that the collimation devices may be used for other types of radiation therapy. FIG. 1 illustrates an example radiotherapy system 100. The system 100 includes radiation source 102, which generates beams of ionizing radiation (e.g., photons, x-rays, etc.) or charged particles (e.g., electrons, protons, carbon ions, or other charged particles). Radiation machines and particle accelerators (i.e., radiation source) for delivering radiation therapy are known in the art and therefore not described in further detail herein. In FIG. 1, the radiation source 102 can generate a beam of electrons 104 that travel towards a subject 120 along an electron beam axis 104a. The beam of electrons 104 can include electrons with enough energy to damage or destroy the tissue that they interact with or pass through. Therefore, the beam of electrons 104 can be used to damage or destroy cancer tissue, for example, a tumor 124 located in the otherwise healthy tissue 122 of the subject 120.

Because the electrons can destroy both tumor 124 and healthy tissue 122, protecting healthy tissue 122 from the electron beam 120 is desirable. For example, the shape of the electron beam 104 generated by the radiation source 102 can be different than the shape of the tumor 124 (i.e. the treatment area). Additionally, even if the electron beam 104 is collimated when leaving the radiation source 102, the electron beam can become spread or distorted as it travels toward its target because the electrons in the electron beam 104 can scatter or smear as they pass through air. This effect is also referred to throughout the present disclosure as “scattering” or “smearing.” The further the electron beam 104 travels, the more pronounced this effect can be. Therefore scattering can also change the shape of the electron beam 104 and cause the electron beam 104 to spread out around the electron beam axis 104a.

As shown in FIG. 1, a collimator 110 can be used to form the electron beam 104 into the desired shape for the tumor 124 that is located on a subject 120. The collimator 110 can include an aperture (not shown) that can shape the electron beam 104 into a collimated electron beam 112 that is focused on the tumor 124 and avoids targeting the healthy tissue 122 surrounding the treatment area. In some implementations of the present disclosure, the collimator 110 can be attached in a fixed position relative to the radiation source, and/or in a fixed position relative to the subject 120.

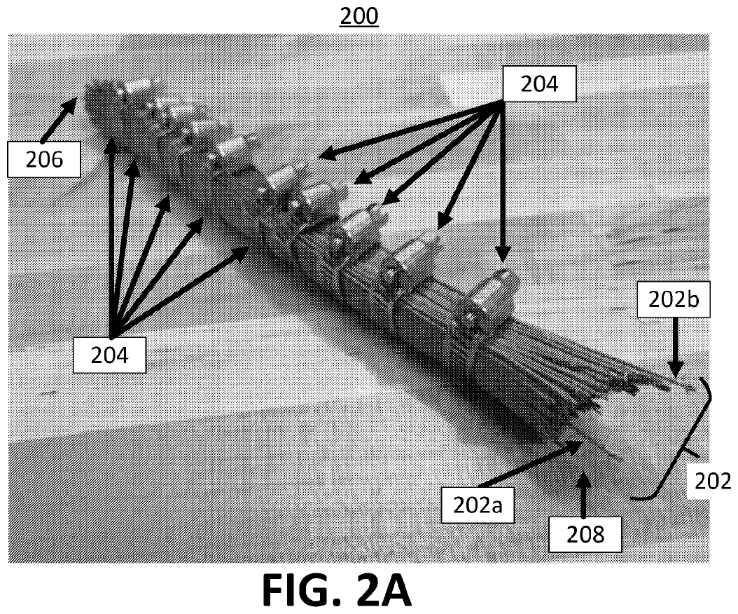

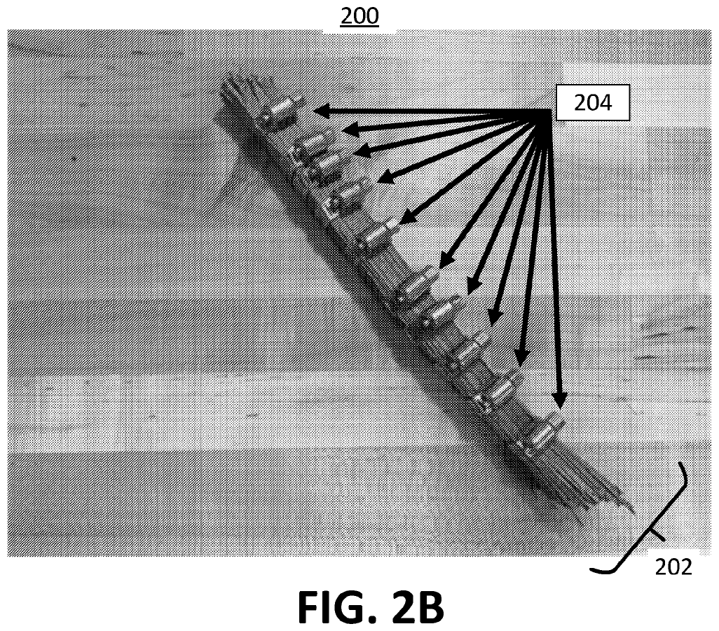

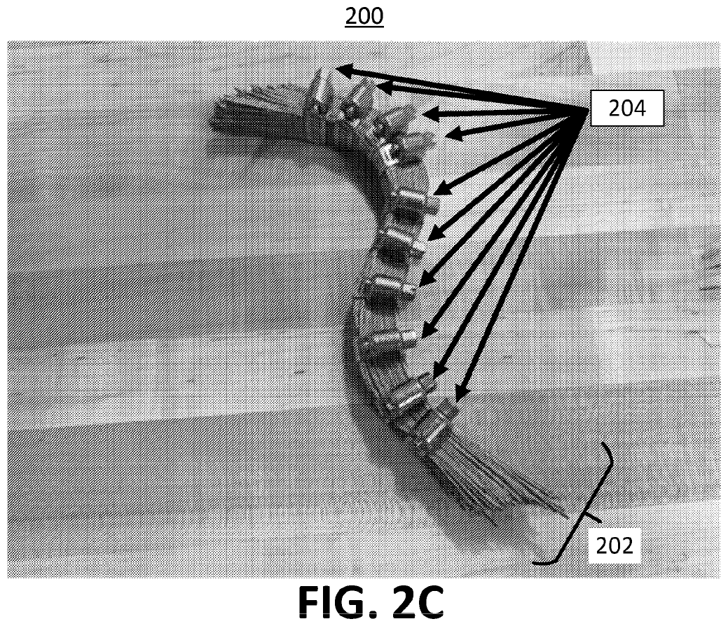

With reference to FIGS. 2A-2C, an implementation of the present disclosure including a flexible collimator 200 is illustrated according to one implementation of the present disclosure. FIG. 2A illustrates a perspective view of the flexible collimator, and FIG. 2B illustrates another perspective view. As shown in FIGS. 2A-2B, the flexible collimator 200 can be used as the collimator 110 illustrated in FIG. 1. The flexible collimator 200 can include any number of elongate members 202a 202b arranged parallel to one another. It should be understood that only two elongate members 202a 202b are labeled in FIG. 2A for illustration purposes. Elongate member(s) are referred to herein individually and collectively as elongate member(s) 202. The collimator 200 can define a first end 206 and a second end 208, and the first end and second end of the collimator 200 can define a longitudinal axis between the first end 206 and second end 208. In some implementations, the longitudinal axis of the collimator 200 can be perpendicular to the beam axis (e.g. the axis 104a illustrated in FIG. 1) along which radiation is delivered to the subject.

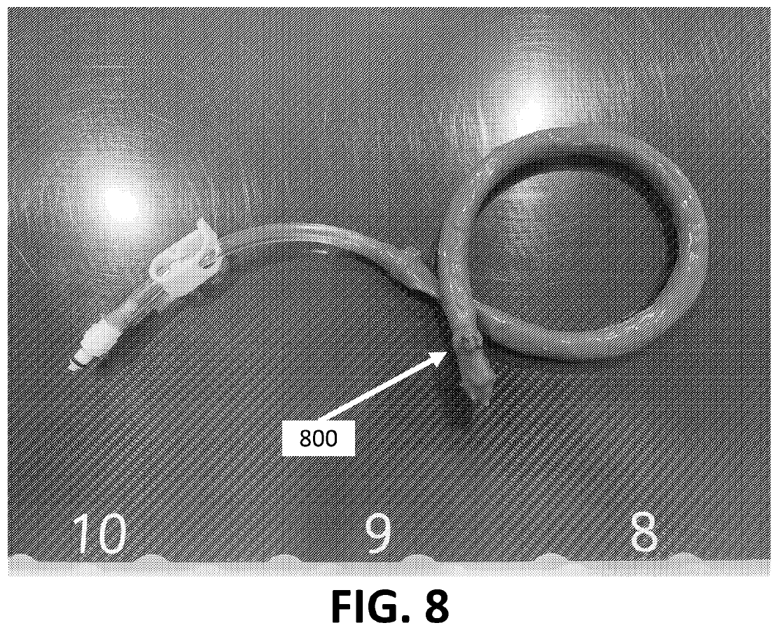

The elongate members 202 can be joined together using one or more fasteners 204. In some implementations, the collimator 200 includes a plurality of fasteners 204 as shown in FIGS. 2A-2C. In other implementations, the collimator includes a single fastener (see e.g., FIG. 8). The fasteners 204 can be used to apply force to keep the elongate members 202 fixed in position relative to one another. When compression is applied, frictional forces keep the elongate members 202 fixed in position relative to one another. This is referred to herein as a “compressed state.” On the other hand, when compression is released, the elongate members 202 are individually slidable relative to one another. This is referred to herein as an “uncompressed state.” In some implementations of the present disclosure, the fasteners 204 at least partially surround the elongate members 202 and can apply pressure to the elongate members 202. In some implementations of the present disclosure, the fasteners 204 can change their size or circumference to apply pressure to the elongate members. As another non-limiting example, the fasteners 204 can optionally be clamps (as shown in FIGS. 2A-2C) where the clamps can be reversibly tightened and loosened to apply varying amounts of compression (and therefore, force) to the elongate members 202. While the clamps shown in FIG. 2A-2C are adjustable using a screw mechanism, it should be understood that other types of clamps, including pneumatic clamps, are contemplated by the present disclosure.

Additionally, in some implementations of the present disclosure, the fasteners can include one or more sections of vacuum-tight sleeves, for example the vacuum tight sleeve 800 illustrated in FIG. 8. The vacuum-tight sleeve 800 can include one more lengths of flexible tubing, and the flexible tubing can partially or completely surround the elongate members. As a non-limiting example, when the fastener is a vacuum-tight sleeve, compression can be applied to the elongate members by decreasing the pressure inside the vacuum-tight sleeve, which results in atmospheric pressure exerting a force on the outside of the vacuum-tight sleeve. When vacuum is applied, frictional forces keep the elongate members fixed in position relative to one another. This is referred to herein as a “compressed state.” On the other hand, when vacuum is released, the elongate members are individually slidable relative to one another. This is referred to herein as an “uncompressed state.” Alternatively, in some implementations of the present disclosure, the fastener can be configured to apply pressure to the elongate members 202 in response to a positive air pressure being applied to the fastener.

When one or more of the fasteners 204 are loosened, some or all of the elongate members 202 can be movable relative to one another. Conversely, when one or more of the fasteners 204 are tightened, the elongate members 202 can be fixed in place relative to one another due to frictional forces. Additionally, it should be understood that in some implementations (like the flexible collimator 200 shown in FIGS. 2A-2C) the fasteners 204 can be individually tightened and loosened to form the elongate members 202 into different shapes. FIG. 2C illustrates the flexible collimator of FIGS. 2A-2B, where the fasteners 204 have been adjusted to reduce compression on the elongate members 202, and the flexible collimator 200 has been contorted, and then the fasteners 204 have been tightened to keep the flexible collimator 200 in that shape shown in FIG. 2C. When the fasteners 204 are tightened to keep the flexible collimator in a shape, that can be referred to as a “compressed state” (e.g. the state illustrated in FIG. 2C). Similarly, when the fasteners 204 are loosened so that the flexible collimator can be adjusted, that can be referred to as an “uncompressed state.” The compressed state can correspond to when the flexible collimator is inflexible, and the uncompressed state can correspond to when the flexible collimator is flexible.

Different implementations of the present disclosure can have different numbers of fasteners, or combinations of different types of fasteners. Additionally, in some implementations of the present disclosure, the fasteners 204 are equally spaced from one another, while in other implementations, some or all of the fasteners 204 can be irregularly spaced from one another.

Implementations of the present disclosure can also include flexible collimators that can be adjusted in different dimensions. As described herein, the flexible collimator 200 can be contorted to surround or partially surround a treatment region such as a tumor. FIG. 2C illustrates a flexible collimator 200 that is adjusted in only two-dimensions (bent into a curved shape on a flat surface). It should be understood that other two-dimensional shapes are contemplated by the present disclosure. As a non-limiting example, the flexible collimator 200 shown in FIGS. 2A-2C could be bent into any two-dimension shape, such as a spiral, circle, or other shape. Additionally, it should be understood that the dimensions of the flexible collimator 200 illustrated in FIGS. 2A-2C are only intended as non-limiting examples. Flexible collimators 200 constructed according to other implementations of the present disclosure can be longer/shorter, thinner/thicker. Additionally, the individual elongate members 202a 202b shown in FIG. 2A can be different sizes/shapes and made from different materials that can allow for different shapes to be formed using the flexible collimator 200. In some implementations of the present disclosure, the elongate members 202 can be formed out of one or more sections of metal cable.

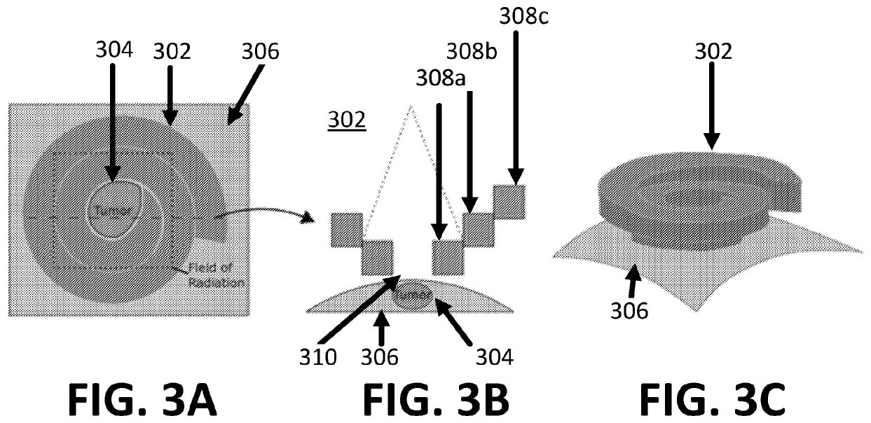

Additionally, the present disclosure contemplates that the elongate members 202 can be shaped to avoid gaps between adjacent cables. Some non-limiting examples of elongate member shapes include tongue-and groove overlaps, as well as concave and convex arcs. For example, each elongate member 202 can have one or more tongue joints, and one or more groove joints, so that, when the elongate members are adjacent to one another the tongue joints of at least some elongate members fit at least partially inside the groove joints of at least some other elongate members. Additionally, in implementations where the elongate member 202 are coiled or spiraled (e.g., as described with reference to FIGS. 3A-3C) the tongue and groove (or concave/convex) surfaces of the same elongate member 202 can be joined together. Additionally, in some implementations, additional fasteners 204 can be added to join adjacent elongate members to one another, in addition to the fasteners 204 that join all the elongate members together.

In some implementations of the present disclosure, the collimator 200 includes a locking mechanism. The locking mechanism can be used to connect a first end 206 of the collimator 200 to the second end 208 of the collimator, or to connect the collimator 200 to another part of a system (e.g. to the radiation source 102 illustrated in FIG. 1), or to connect the collimator 200 to another collimator. In some implementations of the present disclosure, the locking mechanism can be used to connect either the first or second end of one collimator to the first or second end of a second collimator, so that any number of collimators can be used in combination. Additionally, in some implementations, multiple collimators 200 can be spooled together to form a combined collimator where multiple collimators 200 can be spooled together. In some implementations of the present disclosure, the elongate members 202 of one collimator can be shaped to fit with the elongate members 202 of another collimator.

Implementations of the present disclosure can also form three-dimensional shapes and be fixed into three dimensional shapes, as shown in FIGS. 3A-3C. The collimator shown in FIGS. 3A-3C can include a plurality of elongate members and one or more fasteners as described above with reference to FIGS. 2A-2C. FIG. 3A illustrates a top view of a collimator 302 that is fixed in a helix or coiled shape over a tumor 304 located in healthy tissue 306. FIG. 3B illustrates a cutaway side view of the collimator 302 coiled so that the turns 308a 308b 308c of the flexible collimator are not in the same plane. As described with reference to FIGS. 2A-2C, the turns 308a 308b 308c can include locking mechanisms, tongue and groove joints, and/or other features to lock and/or fit adjacent turns 308a 308b 308c together and reduce the radiation flow through the turns 308a 308b 308c. Based on the way that the flexible collimator 302 is adjusted, the shape of the aperture 310 formed by the coiled collimator 302 is a shape corresponding to the tumor 304. FIG. 3C illustrates a perspective view of the coiled collimator 302 placed on the healthy tissue 306. Placing the coiled collimator 302 close to the healthy tissue 306 can minimize the scattering of electrons passing through the aperture 310 and into healthy tissue 306. Additionally, the coiled collimator 302 can optionally be placed in direct contact with the subject's skin.

Additionally, in some implementations of the present disclosure, a locking mechanism can be used to lock the collimator in a particular shape by locking one portion of the elongate members 202 to another portion of elongate members 202 (e.g., the helix or coiled shape described with reference to FIGS. 3A-3C).

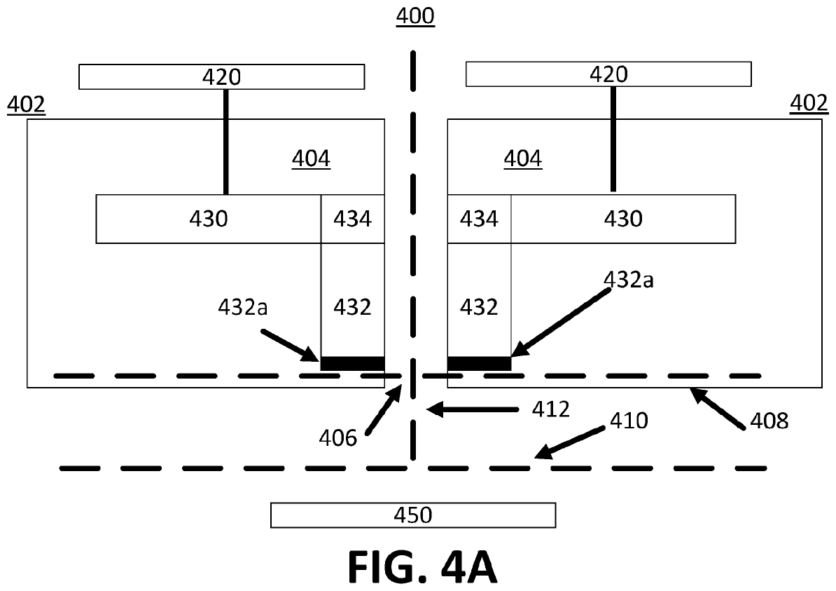

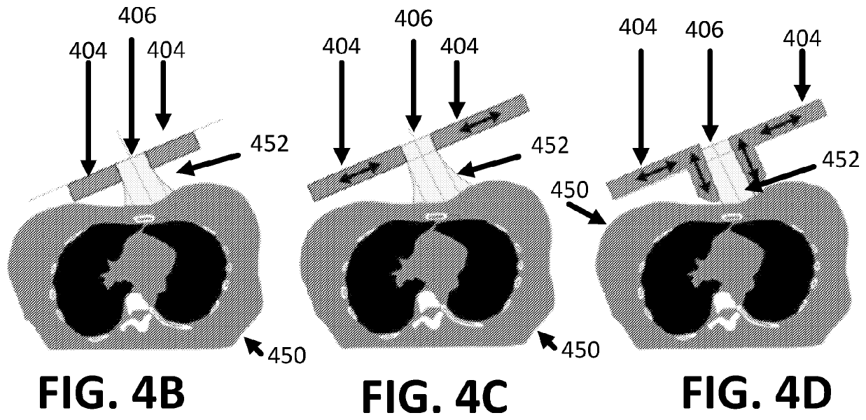

With reference to FIG. 4A, some implementations of the present disclosure include multi-leaf collimators 400. A multi-leaf collimator 400 can include a leaf guide 402. The leaf guide 402 can control the position and orientation of the individual leaves 404 that make up the multi-leaf collimator 400. The leaves 404 can form an aperture 406 where radiation can pass through the collimator. Additionally, the leaf guide 402 can define an aperture plane 408, a first axis 410 and second axis 412. The first axis 410 is perpendicular to the second axis 412, and the second axis 412 is perpendicular to the aperture plane 408.

Still with reference to FIG. 4A, the position and orientation of the plurality of leaves 404 can controlled by the leaf guide 402, and in some implementations of the present disclosure the leaves 404 are partially or completely inside the leaf guide 402. The leaves 404 can be controlled by one or more leaf drivers 420. In some implementations of the present disclosure, a leaf driver 420 can individually move one of the plurality of leaves 404 individually to form the shape of the aperture 406. In other words, each individual leaf 404 has a respective leaf driver 420. In some implementations of the present disclosure, a leaf driver 420 can move two or more of the plurality of leaves 404 to form the shape of the aperture 406. In other words, multiple leaves 404 have a respective leaf driver 420. It should be understood that implementations of the present disclosure can have any number of leaf drivers 420, and that in different embodiments of the present disclosure different numbers of leaves 404 can be controlled by each leaf driver 420.

Additionally, in some implementations of the present disclosure, the leaf driver 420 can move the leaves 404 in more than one direction (e.g., along more than one axis). For example, in the implementation shown in FIG. 4A, the leaves 404 can be moved by one or more leaf drivers 420 along the second axis 412. In FIG. 4A, the second axis 412 is normal to the subject 450, and the first axis 410 is tangential to the subject 450. Alternatively or additionally, in the implementation shown in FIG. 4A, the leaves 404 can be moved by one or more leaf drivers 420 along the first axis 410 and the second axis 412. In either of these implementations, one or more of the leaves 404 is moveable along the second axis 412 to extend beyond the aperture plane 408 in a direction toward the subject 450 as shown. Again, in some implementations of the present disclosure the leaves 404 individually from one another by the leaf drivers 420, so, as non-limiting example, one of the leaves 404 can be at a different location along of the first axis 410 and second axis 412, when compared to the leaves 404 adjacent to it.

Still with reference to FIG. 4A, the leaves can have different shapes. As shown in FIG. 4A, the leaves can include a first section 430, a second section 432, and an angled section 434 that joins the first section 430 and second section 432. The angled section 434 can a curve of any angle or radius, and can also include an angle or corner between the first section 430 and second section 432. As a non-limiting example, the angled section can define a 90-degree angle between the first section 430 and second section 432. As shown in FIG. 4A, the angled section 434 can include an angle that causes the second section 432 to protrude along the first axis 410 in the direction of the subject 450. Additionally, the leaf driver 420 can be configured to move the leaves toward the subject 450, which can improve the collimation of electron travel along the first axis 410 and better control the flow of radiation. As shown in FIG. 4A, the distal tip 432a of the leaves 404 is positioned in the direction of the subject 450, and in some implementations of the present disclosure the leaf driver 420 can position the distal tips 432a of the leaves 404 so that they are touching the subject 450 or are immediately adjacent to the subject 450.

FIGS. 4B-4D illustrate how the leaves 404 can be used to perform collimation. In FIGS. 4B and 4C, an implementation is shown where the leaves 404 are only moved along one axis (i.e., the axis that is tangential to the subject), and do not include a section protruding toward the patient. The beam 452 therefore smears after it passes through the aperture 406. In FIG. 4D, an implementation is shown including leaves with a first and second section, as illustrated in FIG. 4A. In this implementations, the leaves 404 are moved along two axes (i.e., axes that are tangential and normal to the subject). As a result, the leaves 404 have been adjusted to extend beyond the plane of the aperture 406 in a direction toward the subject 450 as shown in FIG. 4D. Accordingly, the electron beam 452 is collimated by the leaves 404 in proximity to the subject, and therefore focused on the treatment area.

The leaves 404 can be made using any suitable material. Non-limiting example materials include plastic, steel, aluminum and brass. Additionally, in some implementations, the leaves 404 can be made of biocompatible materials that are non-toxic. Alternatively or additionally, in some implementations, the leaves can be made of materials configured to minimize x-ray production when the material is exposed to an electron beam.

Furthermore, in some implementations of the present disclosure, the material used to form the collimator leaves can be one that reduces x-ray generation, but has a greater density than plastic. Non-limiting examples of materials that can reduce x-ray generation with higher density than plastic include aluminum, steel, and brass. The present disclosure also contemplates that the leaves 404 can be made from more than one material, or that different leaves in the same collimator 400 can be made from different materials.

The collimator 400 illustrated in FIG. 4A can be used in the system illustrated in FIG. 1 as the collimator 110. For example, the second axis 412 can correspond to the electron beam axis 104a illustrated in FIG. 1, and the shape of the aperture 406 illustrated in FIG. 4A can correspond to the shape of the tumor 124 illustrated in FIG. 1. Accordingly, when the leaf driver 420 illustrated in FIG. 4A moves ones of the leaves 404 along the second axis 412, that moves that leaf toward the subject 120.

As shown in FIG. 4A, the collimator can include multiple leaf guides 402, that can be positioned next to each other so that the leaves 404 of the respective leaf guides 402 form the aperture 406. Again, the leaf guides 402 can be operated separately from one another. Furthermore, it should be understood that the collimator 400 can include any number of leaf guides 402.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Implementations of the present disclosure include low-cost flexible radiation collimators. An example implementation of the present disclosure was constructed and tested. With reference to FIGS. 2A-2C, described above, the example implementation includes a set of elongate members 202 that can run parallel to each other with a variable compression system around them. In this example, the elongate members 202 are cables. It should be understood that cables are only provided as example elongate members. With no compression, the elongate members 202 can slide relative to each other. This can enable the collection of elongate members 202 to be flexible and morphed into a radiation collimator shape. When compression is applied, the increase in frictional forces does not allow the cables to slide relative to each other, thus causing the shape to be fixed. This concept is illustrated in FIG. 2C. In the example implementation, compression is applied with fasteners 204, which are hose clamps as non-limiting examples.

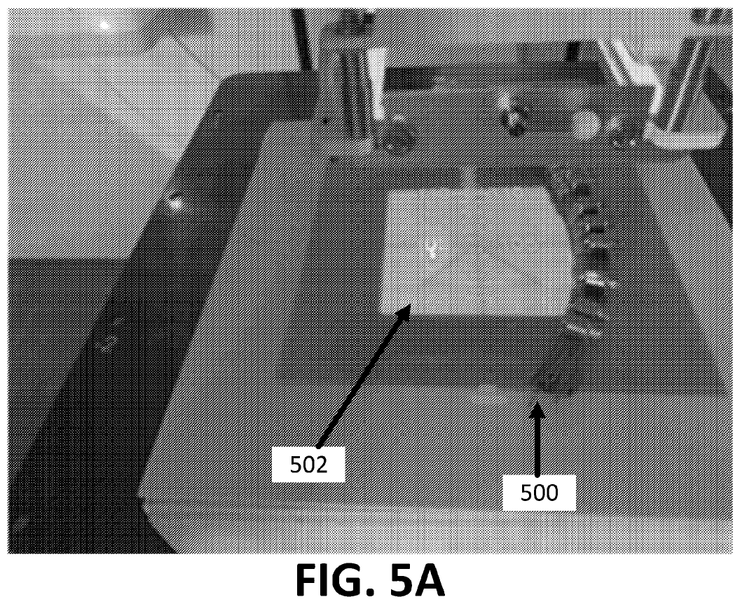

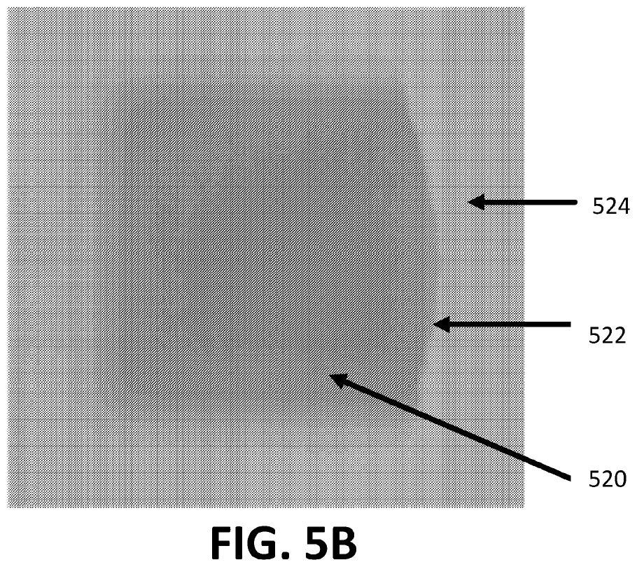

An initial test was performed with radiochromic film to demonstrate the radiation shielding ability of the cable. This film changes its color in response to radiation dose deposition. For the test, a square radiation field of size 10 cm×10 cm was targeted to a block of plastic, with the film on top. FIG. 5A illustrates a setup where the example collimator 500, which includes elongate members and fasteners as described with regard to FIGS. 2A-2C, is placed on a portion of the square radiation field 502. As shown in FIG. 5A, the example collimator 500 was placed on the right side of the field 502. FIG. 5B illustrates the dose falloff corresponding to the dose received using the experimental setup illustrated in FIG. 5A. The radiation dose is shown in FIG. 5B as a shaded region 520, and the sharp edge 522 between the shaded region 520 and the unshaded region 524 illustrates the effectiveness of the shielding of the example implementation illustrated in FIG. 5A. The dose falloff can be seen in the sharpness of the change in color on the film. Additional measurements were performed at deeper depths.

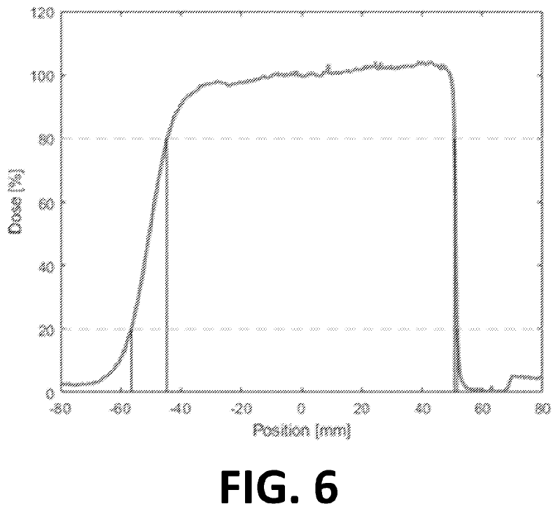

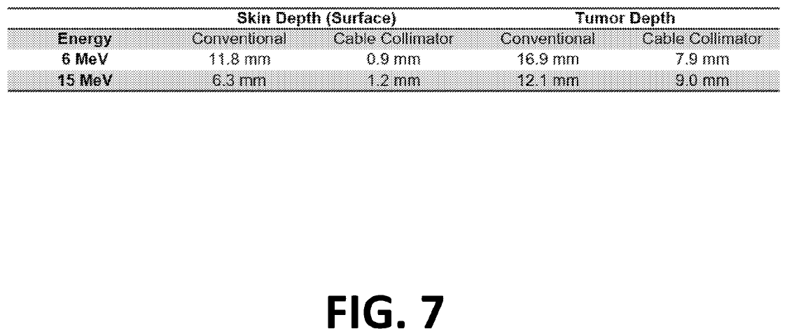

Typical electron beam energies can range from 6 MeV to 20 MeV. The higher energies can scatter less in air and have sharper radiation field edges in comparison to lower energies. The example implementation of a flexible collimator was tested with 6 MeV and 15 MeV. Penumbra is a quantity used to evaluate how fast dose drops outside of a radiation field. More specifically, it can be calculated as the distance it takes radiation to drop from 80% to 20% of the maximum value. FIG. 6 shows the measured radiation (going from left to right through the center of the film) from setup in FIGS. 5A-5B. The difference is most dramatic at the low energies on the surface. The penumbra for the conventional collimator is 11.8 mm. That from the example implementation of the present disclosure is 0.9 mm. FIG. 7 shows a comparison of the calculated penumbra for 6 and 15 MeV at the surface (e.g., skin depth) compared to that at a tumor treating depth. More specifically, the depth used for 6 and 15 MeV are 12 and 27 mm, respectively.

The example collimator 500 used in this example was 20 cm long and demonstrates the ability to lock the cables into a shape. As a non-limiting example, some implementations of the present disclosure can include a longer version of the example implementation that is cable wrapped around itself to create the shape desired for treating the tumor. Additionally, the shape can be warped in three dimensions, which would allow for the inner part of the collimating shape to be as close as possible to the patient, while other parts are farther away. This spooled cable can be connected to a conventional applicator.

The present disclosure also contemplates placing the cables in a vacuum tight sleave. An example of a vacuum tight sleeve 800 that can be used in implementations of the present disclosure is illustrated in FIG. 8. When the sleave 800 is vacuumed, the atmospheric pressure can create the necessary compression.

### Example 2

Implementations of the present disclosure include specially shaped MLCs that can protrude towards the patient and move not only tangential to the patient surface (as other MLCs do and as illustrated in FIGS. 4B and 4C), but also normal to the patient surface (as illustrated in FIG. 4D). This can allow the MLC to be positioned very close to even irregular patient surfaces. This can dramatically improve how sharp dose falls off outside of a target and enables a better radiation sparing of normal tissue adjacent to a target. Beyond this, a practical implementation of an MLC like this could enable multi-energy IMRT for patient treatment.

Implementations of the present disclosure can also be made from plastic. When electrons interact with high Z metals, they can produce x-rays as they slow down. These x-rays can be more difficult to shield than the original electrons. These x-rays can go on and deliver undesirable dose beyond the target (i.e., into the patient or other healthy tissue). Although there can be some x-ray production in plastic interactions, the amount can be much lower than for metals. Making MLCs out of plastic can reduce undesirable radiation and the cost of manufacturing the system.

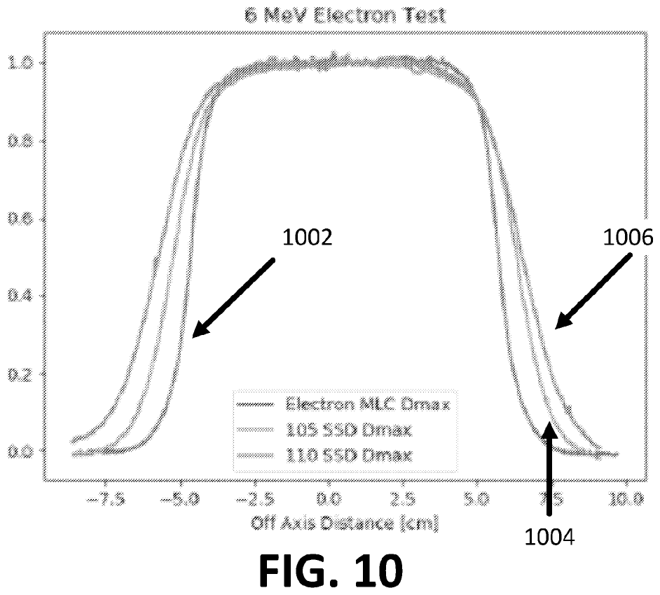

The MLCs can move close to the patient's surface. In the measurements shown in FIG. 10, a safety separation of 2 cm was utilized. This distance is well within the motion accuracy of the example implementation of the MLCs (˜1 mm). Sensing mechanisms can be put on the leaves to fault the system in case of a collision. In some implementations of the present disclosure, the roller bearing system can allow for low friction or almost frictionless MLC motion and low strength motors. The lightweight, plastic MLCs operated using low power motors can be safe to a patient even in the event of collision.

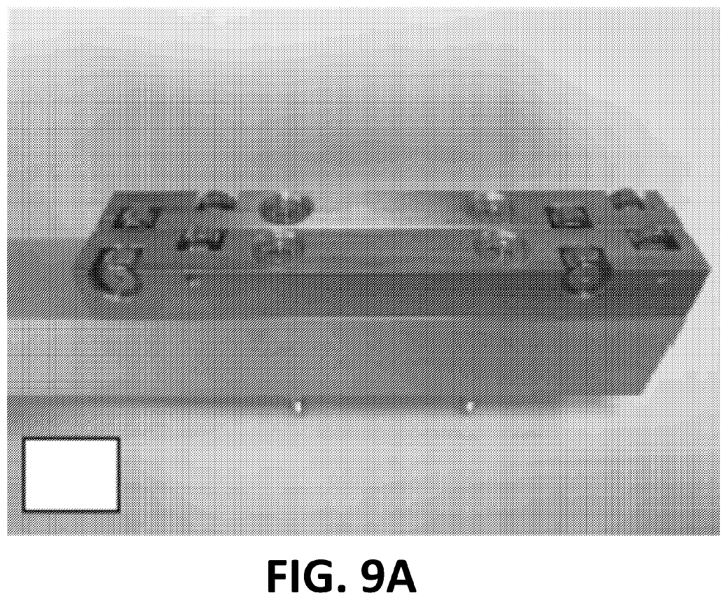

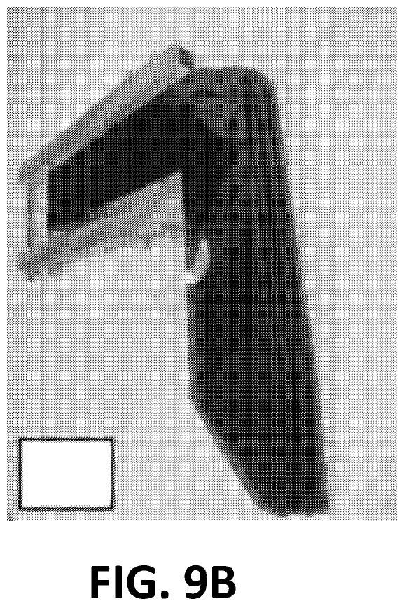

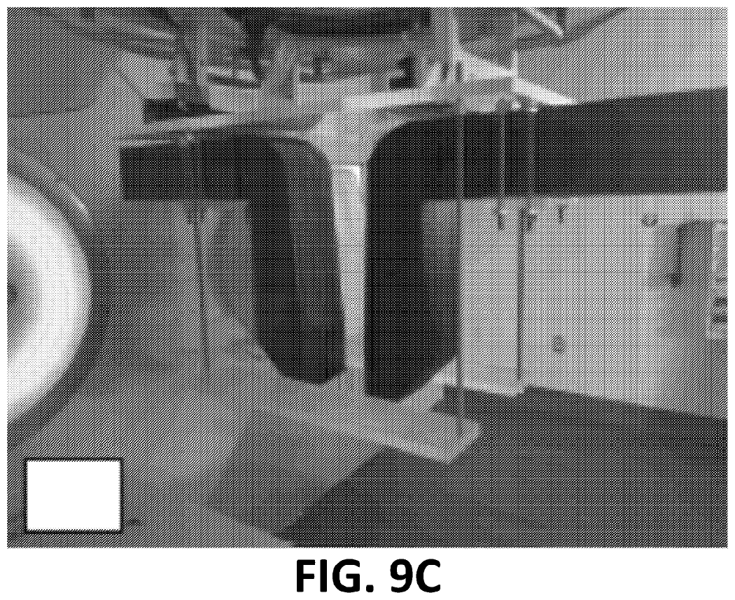

A bench prototype of an implementation of the present disclosure was made and used to generate the data in FIG. 10. The example implementation includes a roller bearing assembly for enabling MLC motion which is illustrated in FIGS. 9A and 9B. The roller bearing assembly bank can have a slim profile that is not wider than the MLCs themselves. This enables adjacent MLC banks to move vertically independent of each other. A fully assembled example MLC system for radiation measurements appears in FIG. 9C. In the example MLC system of FIG. 9C, the surface conforming electron MLC (SCEM) is attached to a linac (replacing a traditional applicator). The leaf banks are surrounded solid water “jaws” that can improve beam collimation in the in-plane axis. In the illustration of FIG. 9C, one of the solid water “jaws” has been removed.

In some implementations of the present disclosure, each MLC can have a motor to move it tangential to a patient surface, similar to the actuation of current MLCs. However, to make the system as practical as possible to operate, MLCs can be grouped into a bank for vertical motion (see FIG. 9B). In the non-limiting example implementation, each bank would include 4 MLCs that would all have the same vertical position. Beyond this, a roller bearing assembly can be used to guide their motion. This can minimize the force necessary to move these leaves and the strength of motors needed to drive them.

Implementations of the present disclosure can allow precise control of dose to tumor versus normal tissue. An experimental implementation was tested and data was acquired. The radiation falloff from the prototype was compared to that of a typical closest electron Cerrobend collimation, 105 cm source to surface distance (SSD), and to another common clinical SSD (110 cm). For simplicity, these two Cerrobend collimation measurements can be viewed as conventional collimation (105 SSD) and that which you might achieve with current state-of-the art MLCs. FIG. 10 illustrates a comparison of the dose falloffs. These dose profiles are taken at a depth similar to that of a tumor and shows how much dose can be spared to adjacent normal tissue. With the ability to produce better dose falloff than current Cerrobend collimation, this can enable IMRT for electron treatments. The dose falloff of the example embodiment is shown as 1002, when compared to a 105 SSD Dmax 1004 and 110 SSD Dmax 1006 dose falloff. Additionally, this can result in a lower integral dose delivered to patients, which is correlated with the production of secondary malignancies. Nguyen F, Rubino C, Guerin S, Diallo I, Samand A, Hawkins M, Oberlin O, Lefkopoulos D, De Vathaire F. Risk of a second malignant neoplasm after cancer in childhood treated with radiotherapy: correlation with the integral dose restricted to the irradiated fields. International Journal of Radiation Oncology⋅Biology⋅Physics⋅ 2008 Mar. 1;70(3):908-15.

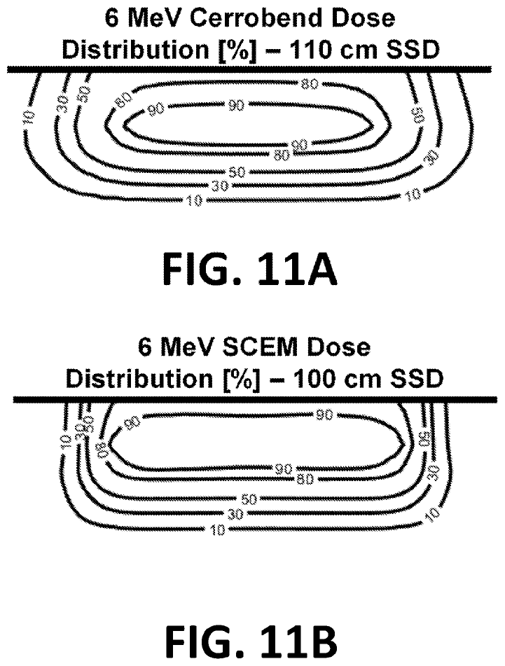

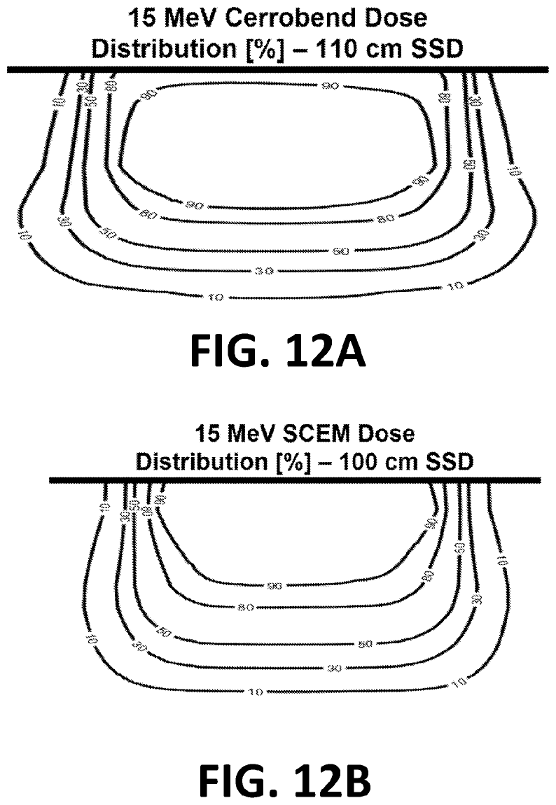

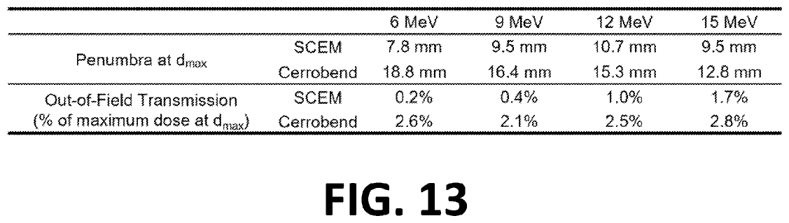

An example implementation of the present disclosure was experimentally tested. FIGS. 11A-12B compare the dose distributions of the SCEM with the Cerrobend cutout for 6 MeV and 15 MeV. FIG. 13 displays a comparison of penumbra and out-of-field dose for the SCEM and Cerrobend cutout. The example implementation of the present disclosure performs conventional collimation in terms of penumbra and reduction of out-of-field dose.

FIGS. 11A-11B illustrate experimental results of a comparison of 6 MeV isodose distributions. Percentages are relative to the maximum dose. FIG. 11A illustrates the isodose distribution for Cerrobend, and FIG. 11B illustrates the isodose distribution of an example implementation of the present disclosure.

FIGS. 12A-12B illustrate experimental results of a comparison of 15 MeV isodose distributions. Percentages are relative to the maximum dose. FIG. 12A illustrates the isodose distribution for Cerrobend, and FIG. 12B illustrates the isodose distribution of an example implementation of the present disclosure.

As illustrated in FIGS. 11A-12B, the SCEM can provide significant improvement in penumbra and reduction of out of field dose. This effect is greatest for 6 MeV and decreases slightly with increasing energy.

FIG. 13 illustrates experimental results of a comparison of penumbra size (defined as the distance between 20% and 80% of central axis dose) and dose transmission 3 cm outside the field edge for the SCEM and Cerrobend cutout for a 10×10 cm field at dmax for different energies.

### Example 3

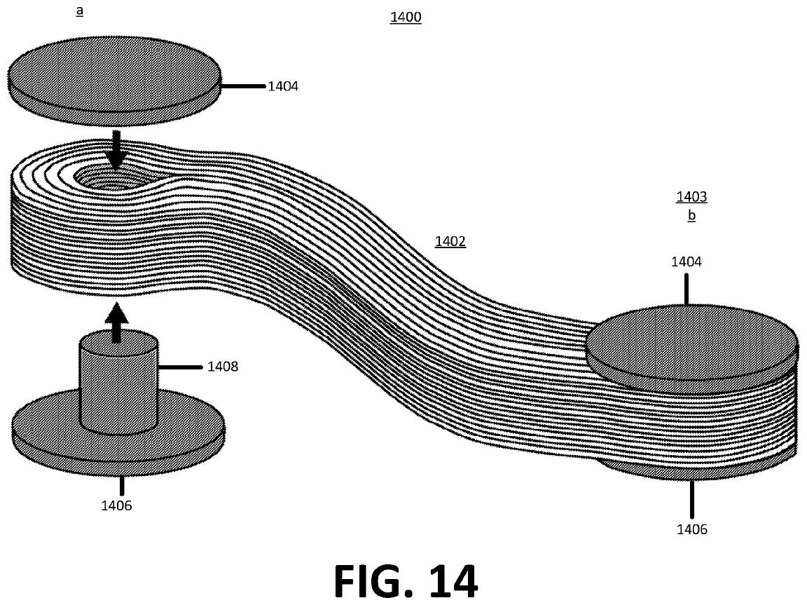

Another example implementation of the present disclosure is illustrated in FIG. 14.

FIG. illustrates a cable collimator 1400. The cable collimator 1400 shown in FIG. 14 can include a number of elongate members 1402. The elongate members 1402 in the implementation shown in FIG. 14 can be arranged so that the elongate members 1402 form a first looped end 1403a and a second looped end 1403b. A hub 1408 can be inserted through the first looped end 1403a and/or the second looped end 1403b. The hub 1408 can be held in place by caps 1404 1406 on either side of the hub 1408. The hub 1408 can be connected to the caps 1404 1406 using an attachable/releasable connection so that the caps can be removed to bend and adjust the elongate members 1402 of the cable collimator 1400. Optionally, the elongate members 1402 can include fewer individual elongate members 1402. In some implementations, the elongate members 1402 can include a single continuous length of cable that is looped repeatedly between the looped ends 1403a 1403b to form the elongate members 1402 in between. The size of the elongate members 1402 and/or hub 1408 and/or caps 1404 1406 can be selected so that the elongate members 1402 can be looped around the hubs 1408 without damaging the elongate members. Optionally, the elongate members 1402 and/or hub 1408 and caps 1404 1406 are sized so that they can clamp to the elongate members 1402 to hold them in place relative to one another. In some implementations, the hub 1408 and/or caps 1404 1406 can include clamping mechanisms to apply pressure to the elongate members 1402.

In some implementations, the hub 1408 and caps 1404 1406 can be made of metals including aluminum, steel, or brass, although the use of any other material is contemplated by the present disclosure.

### Example 4

A study was performed on an example implementation of the present disclosure.

Radiation therapy can be indicated for more than half of all cancer patient treatments.[1] Therapeutic electron beams, typically ranging from 6 to 20 MeV, are can be used for treating superficial tumors due to their limited range in tissue. Despite this advantage, it is commonly only used for treating lumpectomy cavities and cancers affecting the skin. One of the key complications of using electrons for radiotherapy is the fact that they can be easily scattered in air. To combat this issue, the electron beam can be collimated close to the patient's surface using applicators or cones. However, electron applicators do not conform to the surface and leave non-negligible air gaps, allowing electrons to scatter and deposit dose outside of the intended target. Furthermore, patient-specific collimation inserts, or “cutouts”, are often made from a low-temperature casting metal, Cerrobend. Cerrobend is made from toxic metals such as lead and cadmium and is classified as a level-4 health hazard (extreme danger) by the National Fire Protection Association. Cerrobend is carcinogenic and prolonged exposure can cause irreversible damage to the kidneys, liver, skeletal structures, and central nervous system.

Skin collimation is a technique that is sometimes used to reduce scatter dose from electrons. Lead is placed directly on the patient's surface, significantly reducing out-of-field dose.² In addition to surface-blocking techniques, bolus is used to improve the utility of electron therapy. For this, an amount of material is placed on the patient surface to modify the electron range. In its simplest implementation, a thin slab of material will be placed over a treatment area and used to alter the range to a desirable stopping position. Electron conformal radiotherapy (ECRT) is a more advanced application of bolus. For this, three-dimensional printed boluses are custom-tailored for a patient to conform the electron radiation to the distal tumor boundary. There can be minimal bolus in locations where the tumor is the deepest and conversely thick bolus will overlay shallow tumor locations. ECRT enables electrons to treat a variety of other sites: post-mastectomy chest wall, head and neck, and paraspinal muscles.[3-7] One complication with this technique is that the thick areas of this bolus can have a similar effect as a large air gap. It will blur out the edge of an electron beam. Thus, it can sacrifice dose falloff laterally for range modulation ability.

Modulated electron therapy (MERT) is another technique that has been applied to improve the utility of electrons. In this technique, MLCs are used to shape the electron fluence. In contrast to ECRT, range modulation is achieved by mixing electron energies. One technique for this is to position a patient close to a linear accelerator head and collimate with the photon MLCs.[8-11] Another approach has been the use of add-on MLCs.[12-15]. Due to patient collision issues, the collimating material of these previous MLC-based techniques must be further away from the patient surface than conventional applicators. This placement issue fundamentally increases the amount of radiation scattered to normal tissue and limits the ability of these techniques to produce sharp dose falloffs.

However, even with these limitations, MERT has still shown the potential to treat breast, chest wall, and scalp tumors while sparing adjacent healthy tissue better than photon therapy.[16-18]. Eldib et al. studied partial scalp radiotherapy and found that MERT reduced the mean brain dose by 59% compared to photon IMRT treatments.[18] Ma et al. demonstrated a reduction in the maximum heart and lung dose by 20 Gy for MERT compared to photon breast treatments.[17]. Gauer et al. displayed a 35% (2.2 Gy) reduction in the mean heart dose with MERT compared to whole breast conventional photon therapy.[16] Major coronary events are found to increase linearly (with no threshold) with the mean heart dose at a rate of 7.4% per Gy.[19] Thus, it is anticipated that this 2.2 Gy reduction in mean heart dose would lower major coronary events for these patients by 16.3%. Given that there are over two million breast cancer cases per year worldwide, this reduction can have a profound global health impact.²⁰

It can be critical to control radiation delivered not only to tumors and adjacent structures but also the low levels that are scattered elsewhere in the body. Low levels of radiation exposure increase the risk of secondary malignancies. The Women's Environmental, Cancer, and Radiation Epidemiology (WECARE) study evaluated the risk of developing secondary cancer from breast radiotherapy.[2]. They found that women less than 40 years old, who were exposed to more than 1 Gy of radiation to the contralateral breast, had a 2.5-fold increase in risk compared to unexposed women. This dose is only 2% of a common breast irradiation prescription, 50 Gy. For this reason, a radiotherapy strategy that reduces these low levels of scattered radiation has the potential to significantly impact the risk for radiation-induced malignancies

Ultimately, a way to reduce this toxicity is by lowering the radiation dose received by normal tissue. The ideal dose distribution can have a sharp falloff in the penumbra to spare lateral tissue, modulate its range to spare tissue beyond the target, and minimize dose in the tail of the distribution to reduce the potential for secondary malignancies. Skin collimation, ECRT, and MERT techniques have a variety of strengths, but none possess all these ideal characteristics. Implementations of the present disclosure can reduce normal tissue toxicity by an MLC system that conforms to the patient surface. This surface-conforming electron MLC (SCEM) can produce large reductions in normal tissue doses found with MERT, while also minimizing low levels of radiation exposure throughout the body. In the context of breast radiotherapy, this can reduce the risk for inducing both major coronary events and secondary malignancies. In addition to dosimetric advantages, an MLC system can avoid patient-specific fabrication and handling of Cerrobend. The concept for the SCEM device is for leaves to protrude towards the patient and move not only tangential to the patient surface (as other MLCs do), but also normal to the patient surface.

FIGS. 4B through 4D display a comparison of conventional collimation and other MLC approaches to the SCEM strategy. The 2-dimensional motion allows the MLCs to be positioned close to even irregular patient surfaces. This dramatically improves how sharp dose falls off outside of a target and enables better radiation sparing of adjacent normal tissue. Also, these MLCs are made of plastic, which produces less bremsstrahlung than metal collimation. The study described herein included constructing an SCEM prototype for evaluation of the concept. It has leaves that protrude toward the surface (as shown in FIG. 4D); however, the example implementation of the prototype does not have MLC motion normal to the surface. Instead, the dosimetric advantages can be investigated with flat phantom geometries.

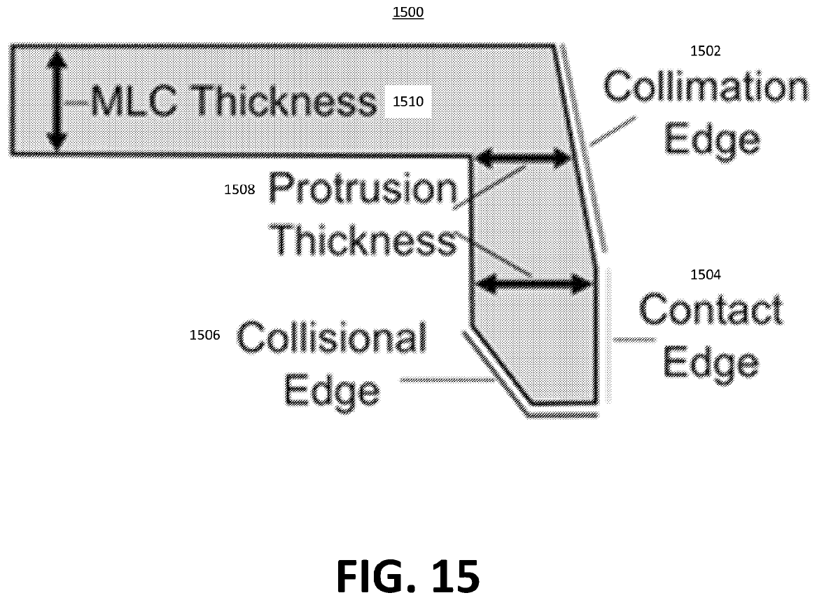

MLC leaves were cut into the shape of the example MLC leaf 1500 shown in FIG. 15. The MLC leaf 1500 includes a collimation edge 1502, a contact edge 1504, a collisional edge 1506, and a protrusion 1508, as shown in FIG. 15. It should be understood that the geometry shown in FIG. 15 is only a non-limiting example Electrons with energy of 15 MeV have a range of 6.3 cm in acrylic. The MLC thickness 1510 and contact edge 1504 were made to be 8 cm to minimize intra-and inter-MLC transmission (when opposing MLCs touch). The collimation edge 1502 was tilted relative to the contact edge 1504 to avoid unnecessary clipping of electrons on a trajectory to the treatment site. The protrusion thickness was 8 cm near the contact edge but tapered down to 7 cm at its smallest (due to this tilt). The outside corner of the MLC protrusion 1508 was cut to make the collisional edge 1506. This can enable the MLC to be as close as possible to an irregular patient surface.

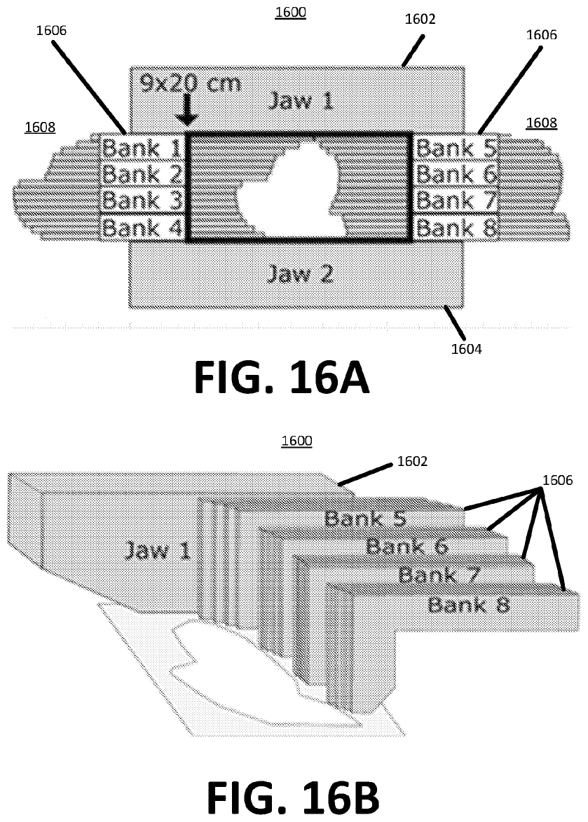

FIG. 16A illustrates a beam's eye view of the SCEM 1600 including bank 1606 with jaws 1602, 1604, and FIG. 16B illustrates a side view of an SCEM including a bank with the jaws. FIGS. 16A and 16B show an SCEM 1600 where a first jaw 1602 and a second jaw 1604 are positioned on either side of banks 1606 of leaves 1608. Optionally, the leaves 1608 can be the MLC leaves 1500 illustrated and described with reference to FIG. 14. In the example shown in FIG. 16A-16B, the width of the bank 1606 of leaves can be fixed a 9 cm in the in-plane direction and the leaves can open to a maximum of 20 cm in the cross-plane direction. A total of 32 of the MLCs were made to test the example implementation, with 16 being put into each opposing bank, as seen in FIGS. 16A-16B. The example MLCs were 5.8 mm in thickness, meaning that 16 leaves together were approximately 9 cm thick.

Implementations of the example device can include grouping MLCs 1600 into smaller banks (Bank 1-8 in FIG. 16A), where each can be able to move normal to the surface independent of the others. An MLC support structure was fabricated on a block tray fixture. This support structure allows for the MLCs to be pulled back as far as 10 cm from the central axis and as far forward as 5 cm beyond it. This allowed the MLC to make field sizes as large as 9×20 cm. In some embodiments, of the device shown in FIG. 16A-16B, the device can also use jaws to terminate the MLC field at each end (see FIG. 16A-16B). In the example implementation, 30×30×5 cm solid water slabs are used as these jaws. FIG. 9C 4 displays the SCEM attached to the head of the linac with one solid water jaw removed to better visualize the leaves. It should be understood that the example banks 1606, Jaws 1602 1604, and leaves 1608 shown in FIGS. 16A-16B are intended only as non-limiting examples in the example implementation that was tested. Any number and combinations of banks 1606, jaws 1602 1604, and leaves 1608 can be used in various implementations of the present disclosure.

### Example Measurements

Water tank scans were taken using a PTW BEAMSCAN (PTW, Freiburg) with the PTW 60012 Diode. Cerrobend cutouts were created for three field sizes (3×9 cm, 5×9 cm, and 10×9 cm) that were defined at the surface. Measurements for the cerrobend cutouts were taken at 100 cm SSD and 110 cm SSD (a more clinically relevant SSD) for all available energies on the Elekta Versa HD linear accelerator (6 MeV, 9 MeV, 12 MeV, and 15 MeV). Percent depth dose (PDD) curves were measured for each energy and for each field size and included depths several centimeters beyond the practical range (Rp) to better characterize the bremsstrahlung tails. Cross-plane profiles were taken at a variety of depths ranging from near-surface (i.e. 1 mm) to depths beyond the practical range. Additionally, cross-plane profiles were scanned several centimeters beyond the field edges to characterize out-of-field dose.

The SCEM leaves were adjusted to match the field sizes of the cerrobend cutouts at the water surface within 1 mm, and all SCEM measurements were made at 100 cm SSD, creating a 0.5 cm airgap from the tip of the SCEM to the water surface. PDD curves were measured for each energy and field size, and cross-plane profiles were taken at the same depths as the cerrobend cutouts. Output factor measurements were taken in solid water using a PTW Semiflex ion chamber (0.125 cm³) for each energy at dₘₐₓ for both the cerrobend cutouts and SCEM, both at 100 cm SSD. Additionally, output factors were taken with a fixed phantom sized using a MiniPhantom (Standard Imaging, Middleton, WI). The MiniPhantom was oriented horizontally, and was shifted for each energy so that the chamber center would be at the same distance from the source as in the solid water measurements. This allowed for an evaluation of output changes without increased lateral scatter from the phantom material.

### Example Results

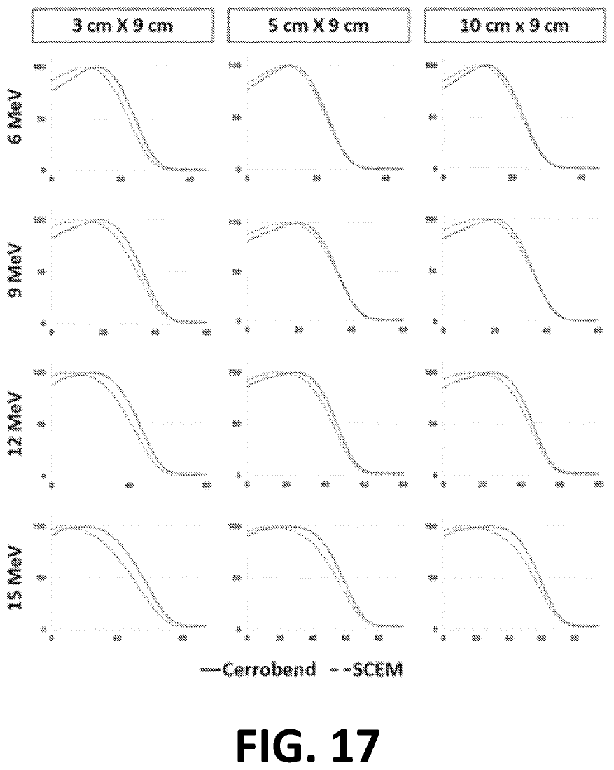

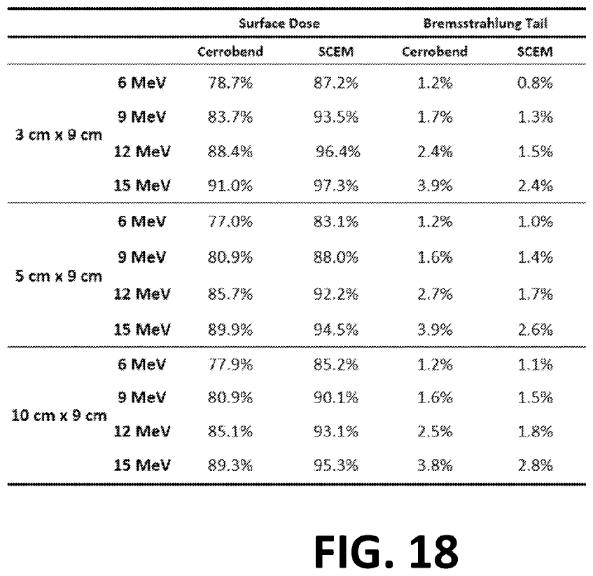

The PDD curves for the SCEM and cerrobend cutouts can be seen in FIG. 17. The PDDs displayed for the cerrobend cutouts were taken at 100 cm SSD, as there was no significant difference from the PDDs for cerrobend cutouts at 110 cm SSD. FIG. 18 summarizes the surface dose (relative to the maximum dose) and the bremsstrahlung tail (i.e. the central axis dose relative to dmax 1 cm beyond the practical range) for the SCEM and the Cerrobend cutout at 100 cm SSD.

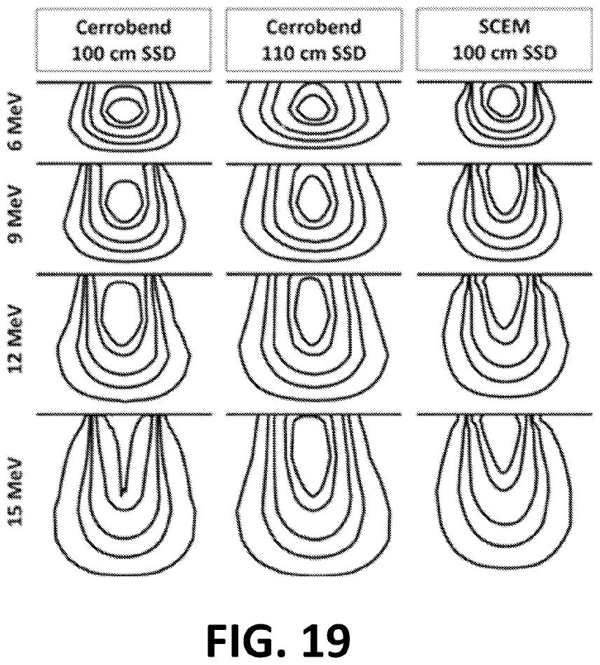

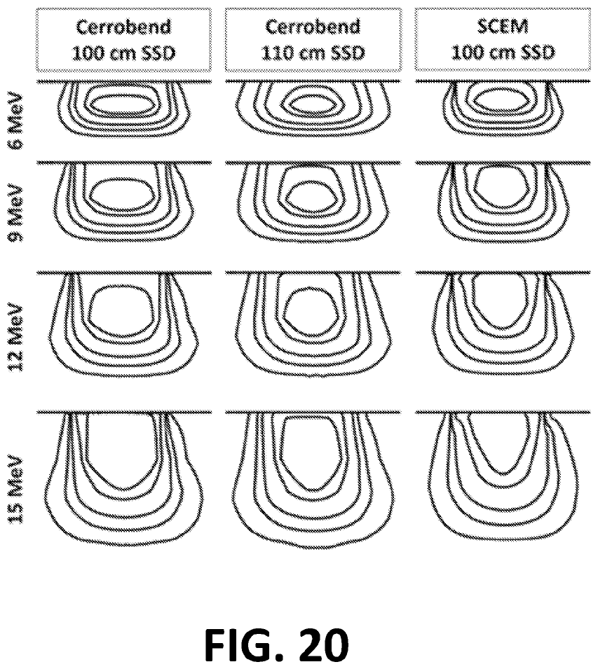

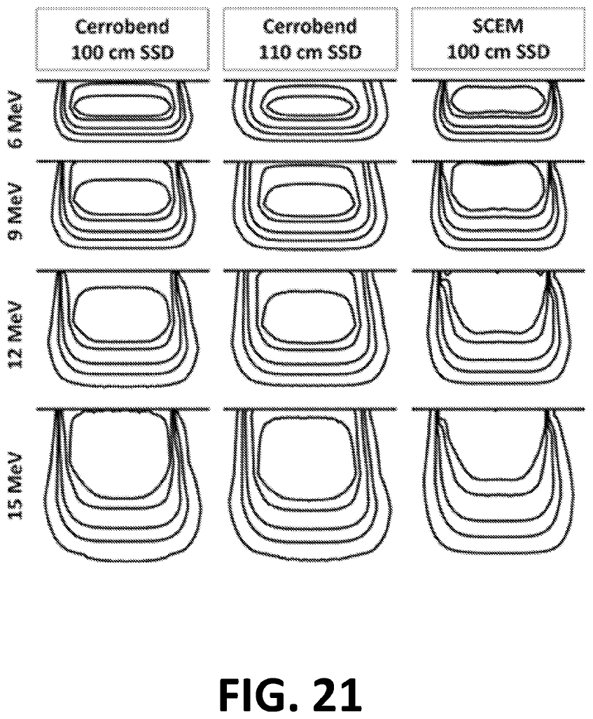

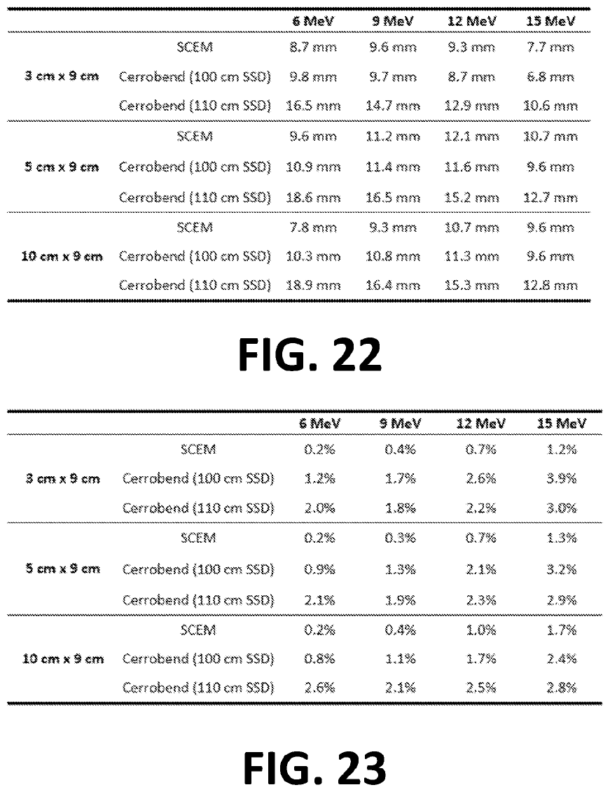

A comparison of the isodose lines for the cerrobend cutout at 100 cm SSD, cerrobend cutout at 110 cm SSD, and the SCEM for the 3×9 cm, 5×9 cm, and 10×9 cm field sizes can be seen in FIGS. 19, 20, and 21, respectively. FIG. 22 displays the penumbra, which is defined as the lateral distance between the 80% and 20% isodose lines at dmax. A comparison of the dose transmission 3 cm from the field edge for the SCEM, and cerrobend cutouts (100 cm SSD and 110 cm SSD) for all energies and field sizes can be seen in FIG. 23.

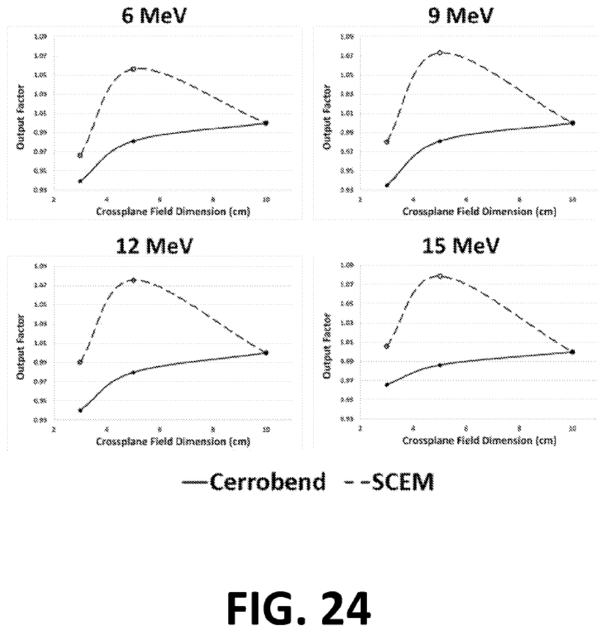

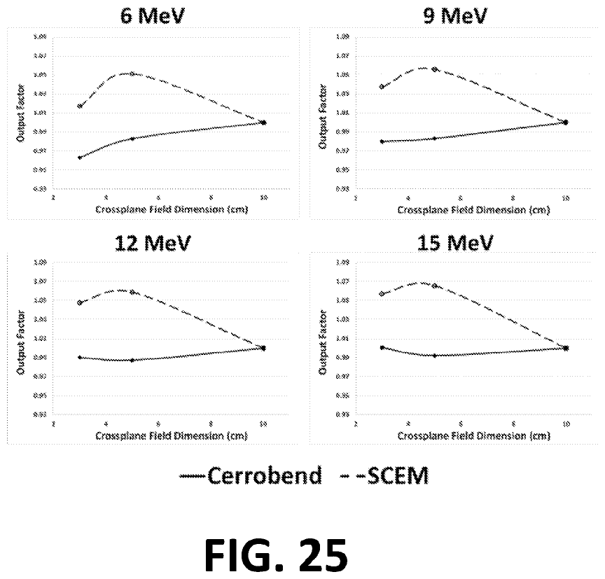

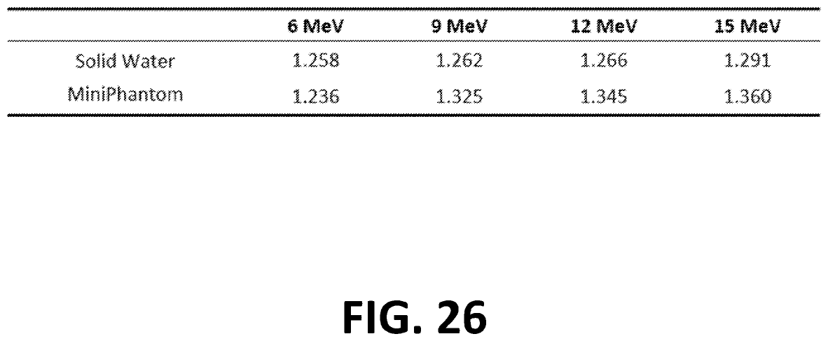

Plots of the output factors taken in solid water (normalized to the 10×9 cm field) can be seen in FIG. 24. The 5×9 cm field showed the highest relative output and increased with increasing energy. The relative output of the 3×9 cm field was less than the 10×9 field for all energies except for 15 MeV, where it was slightly greater. Plots of the output factors taken with the MiniPhantom (normalized to the 10×9 cm field) can be seen in FIG. 25. Above 6 MeV, the output for the Cerrobend cutout remain relatively constant, varying less than 2% from the output of the 10×9 cm field. The SCEM output for the smaller field sizes (3×9 cm and 5×9 cm) varied considerably when compared to the 10×9 cm field, with this effect becoming more pronounced with increasing energy. The highest relative output was seen for the 5×9 cm field at 15 MeV, where it was approximately 7% higher than the 10×9 cm field. It is also worth noting that the raw measured charge readings were much higher for the SCEM than for the Cerrobend cutouts. FIG. 26 displays the ratios of raw charge readings (SCEM to Cerrobend cutout) for the 10×9 cm field for all energies for both the solid water and MiniPhantom setups. For both experimental setups, the measured charge readings were at least 23.6% greater for the SCEM than for the Cerrobend cutouts for all energies.

Implementations of the present disclsoure include an electron collimation system that combines the advantages of skin collimation, ECRT, and MERT. The initial SCEM protoype provides an advantage over lead skin collimation in that it eliminates the need to cut lead for individual patients. A disadvantage of the current SCEM design when compared to lead skin collimation is that there is the potential for collisions with the patient or immobilization equipment. Additionally, lead skin collimation allows the physician to directly place the lead on the area they want to shield and allows for better visualization of the area that will be treated. When compared to custom-printed bolus, the SCEM can save time by elimating the need to 3D print a bolus for individual patients.

As previously discussed, there have been several studies on the use of photon MLCs.⁸⁻¹¹ While the use of photon MLCs for electron treatments allows electron fields to be more easily combined with photon fields (thereby eliminating the need for treatment interruption), there are several disadvantages. Photon MLCs have been shown to have bremsstrahlung contamination comparable to conventional Cerrobend cutouts, thereby providing little to no benefit in the reduction of out-of-field dose and photon contimination.[8] Because the SCEM is made of acryllic, the bremsstrahlung production is significantly reduced, thereby reducing the out-of-field dose. Additionally, to avoid collisions with a patient, photon MLCs are limited in how close they can be to a patient's surface. This leads to increased dose to organs at risk near the field.¹⁰ The SCEM concept allows for collimation closer to the patient's skin surface, thus reducing the penumbra and dose to areas outside the tumor volume.

Several groups have proposed designs for add-on electron MLCs.[12-15]. While photon MLCs have been shown to increase in-air scattering, an add-on MLC system can allow for the field to be shaped closer to the patient's surface. Previous designs of add-on MLC systems have used source-to-collimator distances (SCDs) comparable to traditional electron applicators.[12, 15] While these designs showed some advantages over photon MLCs, they did not show significant improvement in reducing the penumbra when compared to applicators with Cerrobend inserts, particularly for low energies. The SCEM provides decreased penumbra due to it's ability to collimate almost directly on the surface.

Embodiments of the present disclosure compare the current SCEM design with traditionally used Cerrobend cutouts. For all field sizes and energies, the dmax for the SCEM shifted towards the surface, and this effect was more pronounced with increasing energy. The surface dose relative to dmax for the SCEM was higher than for the Cerrobend cutout for all energies and field sizes, with the largest difference seen at 9 MeV for all three field sizes. The bremsstrahlung tail for the SCEM was lower than Cerrobend for all energies and field sizes. The SCEM showed a significant decrease in penumbra for all energies and field sizes when compared to the Cerrobend cutout at 110 cm SSD, particularly at shallower depths and lower energies. The largest improvement in penumbra for the 10×9 cm field was at 6 MeV, decreasing by 58.8% from the Cerrobend cutout at 110 cm SSD. The out-of-field dose for all field sizes and energies decreased when the SCEM was compared to the Cerrobend cutouts (for both 100 cm SSD and 110 cm SSD), with the largest decrease occurring for the 6 MeV 10×9 cm field.

Changes in the output factor are affected by establishment of lateral equilibrium and also scatter from outside the phantom. The two setups, one with solid water and one with the MiniPhantom, were used to evaluate output changes. The MiniPhantom has a fixed width of 3 cm which allows for an evaluation of output changes without increased lateral scatter with field size. In the MiniPhantom above 6 MeV, the outputs for the Cerrobend cutouts are relatively stable. However, the outputs for the SCEM vary by as much as 7% (for the 5×9 cm field at 15 MeV) when compared to the reference field size of 10×9 cm. Since there is no change in the amount of phantom being irradiated, the large differences in output must be due to the additional scatter off the SCEM surface.

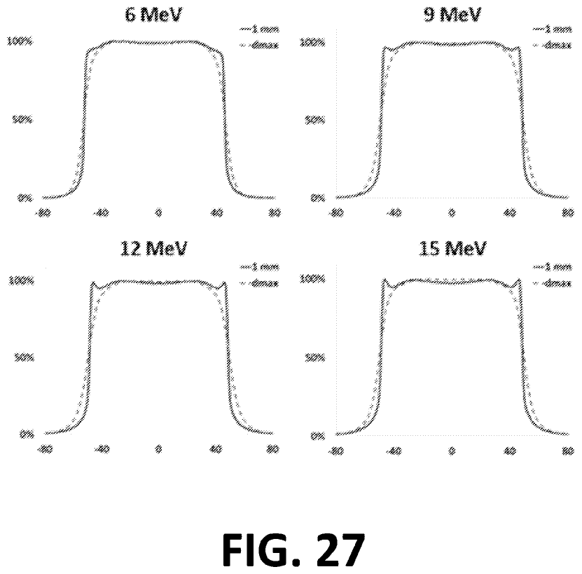

A disadvantage of the SCEM is the “horns” on the shallow depth field edges for higher energies at the largest field size, as seen in FIG. 27. While this effect dimishines with increasing depth, it provides sub-optimal dosimetry near the surface. The “horns” on the field edges are likely to due the larger scattering surface of the SCEM when compared to the Cerrobend cutous. This effect should decrease with increasing the atomic number of the collimating material (i.e. decreasing the thickness needed to collimate the electron beam decreases the scattering surface). Despite this, the SCEM provides several advantages over Cerrobend cutouts. In addition to being toxic and posing various health risks, Cerrobend cutouts must be poured for each field to be treated.

The example implementations of the present disclosure studied can provide several benefits over the current standard of applicators with custom-made Cerrobend inserts. When compared to Cerrobend cutouts, the example SCEM reduced penumbra by up to 58.8%. Additionally, the construction of a low-Z material lowers the bremsstrahlung production significantly, thereby decreasing the out-of-field dose. The SCEM reduced the out-of-field dose by up to 92.3%. Implementations of the present disclosure can optionally include improving sub-optimal dosimetric characteristics of the SCEM, particularly the “horns” seen in shallow-depth profiles at high energies for larger field sizes and the increase in shallow depth PDDs. This can optionally be accomplished using of a different material for the leaves. Additionally, implementations of the present disclosure can include controlling leaf motion in both the tangential and normal directions, as well as the use of the SCEM in modulated electron therapy.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

### REFERENCES

1. Delaney, G., Jacob, S., Featherstone, C. & Barton, M. The role of radiotherapy in cancer treatment: Estimating optimal utilization from a review of evidence-based clinical guidelines. Cancer 104, 1129-1137 (2005).
2. Hogstrom, K. R. & Almond, P. R. Review of electron beam therapy physics. Phys. Med. Biol. 51, R455-R489 (2006).
3. Su, S., Moran, K. & Robar, J. L. Design and production of 3D printed bolus for electron radiation therapy. J. Appl. Clin. Med. Phys. 15, 194-211 (2014).
4. Kudchadker, R. J. et al. Electron conformal radiotherapy using bolus and intensity modulation. Int. J. Radiat. Oncol. 53, 1023-1037 (2002).
5. Kudchadker, R. J., Antolak, J. A., Morrison, W. H., Wong, P. F. & Hogstrom, K. R. Utilization of custom electron bolus in head and neck radiotherapy. J. Appl. Clin. Med. Phys. 4, 321-333 (2003).
6. Craft, D. F. et al. Design, fabrication, and validation of patient-specific electron tissue compensators for postmastectomy radiation therapy. Phys. Imaging Radiat. Oncol. 8, 38-43 (2018).
7. Low, D. A. et al. Computer-aided design and fabrication of an electron bolus for treatment of the paraspinal muscles. Int. J. Radiat. Oncol. 33, 1127-1138 (1995).
8. Plessis, F. C. P. du, Leal, A., Stathakis, S., Xiong, W. & Ma, C. -M. Characterization of megavoltage electron beams delivered through a photon multi-leaf collimator (pMLC). Phys. Med. Biol. 51, 2113-2129 (2006).
9. Klein, E. E., Mamalui-Hunter, M. & Low, D. A. Delivery of modulated electron beams with conventional photon multi-leaf collimators. Phys. Med. Biol. 54, 327-339 (2009).
10. Mueller, S. et al. Electron beam collimation with a photon MLC for standard electron treatments. Phys. Med. Biol. 63, 025017 (2018).
11. Kaluarachchi, M. M., Saleh, Z. H., Schwer, M. L. & Klein, E. E. Validation of a Monte Carlo model for multi leaf collimator based electron delivery. Med. Phys. 47, 3586-3599 (2020).
12. Lee, M. C., Jiang, S. B. & Ma, C. -M. Monte Carlo and experimental investigations of multileaf collimated electron beams for modulated electron radiation therapy. Med. Phys. 27, 2708-2718 (2000).
13. Hogstrom, K. R. et al. Dosimetry of a prototype retractable eMLC for fixed-beam electron therapy. Med. Phys. 31, 443-462 (2004).
14. Gauer, T. et al. Characterization of an add-on multileaf collimator for electron beam therapy. Phys. Med. Biol. 53, 1071-1085 (2008).
15. O'Shea, T. P., Ge, Y., Foley, M. J. & Faddegon, B. A. Characterization of an extendable multi-leaf collimator for clinical electron beams. Phys. Med. Biol. 56, 7621-7638 (2011).
16. Gauer, T., Engel, K., Kiesel, A., Albers, D. & Rades, D. Comparison of electron IMRT to helical photon IMRT and conventional photon irradiation for treatment of breast and chest wall tumours. Radiother. Oncol. 94, 313-318 (2010).
17. Ma, C. -M. et al. A comparative dosimetric study on tangential photon beams, intensity-modulated radiation therapy (IMRT) and modulated electron radiotherapy (MERT) for breast cancer treatment. Phys. Med. Biol. 48, 909-924 (2003).
18. Eldib, A. et al. Investigating the dosimetric benefits of modulated electron radiation therapy (MERT) for partial scalp patients. Biomed. Phys. Eng. Express 3, (2017).
19. Darby, S. C. et al. Risk of Ischemic Heart Disease in Women after Radiotherapy for Breast Cancer. N. Engl. J. Med. 368, 987-998 (2013).
20. Bray, F. et al. Global cancer statistics 2018: GLOBOCAN estimates of incidence and mortality worldwide for 36 cancers in 185 countries. CA. Cancer J. Clin. 68, 394-424 (2018).
21. Stovall, M. et al. Dose to the Contralateral Breast From Radiotherapy and Risk of Second Primary Breast Cancer in the WECARE Study. Int. J. Radiat. Oncol. 72, 1021-1030 (2008).
22. Rusk, B. D., Carver, R. L., Gibbons, J. P. & Hogstrom, K. R. A dosimetric comparison of copper and Cerrobend electron inserts. J. Appl. Clin. Med. Phys. 17, 245-261 (2016).

What is claimed:

1. A radiotherapy collimation device comprising:
a plurality of elongate members arranged in parallel to one another; and
at least one fastener at least partially surrounding the plurality of elongate members, the at least one fastener being configured to apply compression to the plurality of elongate members, wherein:
the plurality of elongate members are individually slidable in an uncompressed state, and
the plurality of elongate members are individually fixed in a compressed state.

2. The radiotherapy collimation device of claim 1, wherein the radiotherapy collimation device is flexible and configured to contort into a collimator shape in the uncompressed state.

3. The radiotherapy collimation device of claim 2, wherein the radiotherapy collimation device is inflexible and fixed in the collimator shape in the compressed state.

4. The radiotherapy collimation device of claim 2 or 3, wherein the collimator shape is a two-dimensional shape.

5. The radiotherapy collimation device of claim 2 or 3, wherein the collimator shape is a three-dimensional shape.

6. The radiotherapy collimation device of claim 5, wherein the three-dimensional shape is a helix.

7. The radiotherapy collimation device of any one of claims 1-6, wherein the at least one fastener is a plurality of fasteners, each of the plurality of fasteners at least partially surrounding the plurality of elongate members.

8. The radiotherapy collimation device of any one of claims 1-6, wherein the at least one fastener is a clamp.

9. The radiotherapy collimation device of claim 8, wherein the clamp is adjustable to change a circumference of the clamp.

10. The radiotherapy collimation device of any one of claims 1-6, wherein the at least one fastener is a vacuum-tight-sleeve.

11. The radiotherapy collimation device of claim 10, wherein the vacuum-tight-sleeve is a flexible tubing, and wherein each of the plurality of elongate members is arranged at least partially inside the flexible tubing.

12. The radiotherapy collimation device of claim 11, wherein the flexible tubing comprises an elastic material.

13. The radiotherapy collimation device of claim 11, wherein the flexible tubing is configured to apply pressure to the plurality of elongate members in response to a change in pressure within the flexible tubing.

14. The radiotherapy collimation device of any one of claims 1-13, wherein each of the plurality of elongate members is a metal cable.

15. The radiotherapy collimation device of claim 14, further comprising a locking mechanism configured to attach to an end of the plurality of elongate members.

16. The radiotherapy collimation device of claim 14, wherein the radiotherapy collimation device has a first end and a second end, each of the first and second ends defined by respective ends of the plurality of elongate members, the radiotherapy collimation device further comprises a locking mechanism configured to attach the first end to the second end of the radiotherapy collimation device.

17. A system for performing radiotherapy comprising:
a radiation source configured to emit a beam of electron radiation along a beam axis toward a subject;
a radiotherapy collimation device according to any one of claims 1-16, wherein the radiotherapy collimation device is configured for forming the beam into a desired beam shape in proximity to the subject.

18. The system of claim 17, wherein the plurality of elongate members define a longitudinal axis, and the longitudinal axis is perpendicular to the beam axis.

19. The system of claim 17 or 18, wherein the radiotherapy collimation device is attached in a fixed position relative to the radiation source.

20. The system of claim 17 or 18, wherein the radiotherapy collimation device is arranged in a fixed position relative to the subject.

21. A multi-leaf collimation device comprising:
a leaf guide defining an aperture plane, a first axis, and a second axis, wherein the first axis is perpendicular to the second axis, and wherein the second axis is perpendicular to the aperture plane;
a plurality of leaves arranged within the leaf guide; and
one or more leaf drivers operably connected to the plurality of leaves, wherein the one or more leaf drivers are configured to move the plurality of leaves to form a radiation-beam aperture in the aperture plane, and wherein at least one leaf is moveable along the second axis to extend beyond the aperture plane in a direction toward a subject.

22. The multi-leaf collimation device of claim 21, wherein the at least one leaf comprises a first section, a second section, and a curved section, wherein the first and second sections are separated by the curved section.

23. The multi-leaf collimation device of claim 22, wherein the curved section defines about a 90 degree angle between the first and second sections.

24. The multi-leaf collimation device of any one of claims 21-23, wherein the one or more leaf drivers are configured to move the at least one leaf along the first and second axes.

25. The multi-leaf collimation device of any one of claims 21-24, wherein the at least one leaf is made of a biocompatible material.

26. The multi-leaf collimation device of claim 25, wherein the at least one leaf comprises plastic.

27. The multi-leaf collimation device of claim 25, wherein the at least one leaf comprises steel, aluminum, or brass.

28. The multi-leaf collimation device of any one of claims 21-25, wherein the at least one leaf is made of a material that minimizes x-ray production when absorbing electrons.

29. The multi-leaf collimation device of any one of claims 21-28, wherein the one or more leaf drivers are configured to move the plurality of leaves independently from each other.

30. The multi-leaf collimation device of any one of claims 21-29, wherein the leaf guide comprises a first leaf guide comprising a first plurality of leaves and a second leaf guide comprising a second plurality of leaves, wherein the one or more leaf drivers are configured to move the first and second plurality of leaves to form the radiation-beam aperture in the aperture plane.

31. A radiotherapy system comprising:
a multi-leaf collimation device according to any one of claims 21-30; and
a radiation source configured to emit a beam of electron radiation along the second axis toward a subject, wherein the multi-leaf collimation device is configured for forming the beam into a desired beam shape in proximity to the subject.

32. A radiotherapy collimation device comprising:
a plurality of elongate members arranged in parallel to one another and forming a plurality of loops; and
at least one fastener configured to apply compression to the plurality of elongate members, wherein:
the plurality of elongate members are individually slidable in an uncompressed state, and
the plurality of elongate members are individually fixed in a compressed state.

33. The radiotherapy collimation device of claim 32, wherein the at least one fastener comprises a hub configured to pass through at least one of the plurality of loops and a cap configured to clamp the hub to at least one of the plurality of loops.
